(19) United States
(12) Patent Application Publication
(10) Pub. No.: US 2019/0276543 A1
(43) Pub. Date: Sep. 12, 2019

(54) COMPOSITIONS AND METHODS FOR TREATING LATE STAGE LUNG CANCER

(71) Applicant: AstraZeneca Pharmaceuticals LP, Wilmington, DE (US)

(72) Inventors: Giovanni Melillo, Wilmington, DE (US); Marc Ballas, Wilmington, DE (US); Simon Dovedi, Cambridge (GB); Ross Stewart, Cambridge (GB)

(21) Appl. No.: 15/915,359

(22) Filed: Mar. 8, 2018

(51) Int. Cl. C07K 16/28; A61P 35/00

(52) U.S. Cl. CPC C07K 16/2827; A61P 35/00; A61K 2039/505; C07K 2317/24; C07K 2317/21

(57) ABSTRACT

Disclosed are methods for treating late stage (e.g., clinical stage III or IV), unresectable non-small-cell lung cancer (NSCLC) with an antibody that inhibits PD1/PD-L1 activity in a patient identified as having not progressed following definitive chemoradiation therapy.

### BACKGROUND

Approximately one-third of patients with non-small-cell lung cancer (NSCLC) have stage III, locally advanced disease at diagnosis. The standard of care for patients with a good performance status (PS) and unresectable stage III NSCLC is platinum-based doublet chemotherapy concurrent with radiotherapy. However, median progression-free survival (PFS) with concurrent chemoradiation therapy (cCRT) in this population is ˜8 months and only 15% of patients are alive at 5 years. In addition, there have been no major advances in this setting in many years. As such, there remains a significant unmet need for novel therapeutic approaches to boost patient survival beyond cCRT.

Programmed cell death ligand-1 (PD-L1) on tumor and myeloid cells in the tumor microenvironment bind to the immune checkpoint protein PD-1 on activated T cells, inhibiting their activity. Durvalumab is a selective, high-affinity, human IgG1 monoclonal antibody that blocks PD-L1 binding to PD-1 and CD80, allowing T cells to recognize and kill tumor cells. Durvalumab has demonstrated encouraging antitumor activity in an early-phase clinical study across multiple advanced solid tumors, and has been approved for post-platinum, locally advanced or metastatic urothelial carcinoma.

In addressing the need for improved methods for clinical management of late stage cancers, the disclosure provides methods comprising administration of durvalumab to patients with late stage, locally advanced, unresectable NSCLC, and whose disease had not progressed following chemoradiation therapy (cCRT). As disclosed herein, the methods provide a significant and unexpected advance to the existing standard of care in patients with late-stage, locally advanced, unresectable NSCLC, who have not responded to chemoradiation therapy (cCRT).

### SUMMARY

The disclosure generally relates to methods for treating late stage (e.g., clinical stage III or IV), unresectable non-small-cell lung cancer (NSCLC) with an antibody that inhibits PD1/PD-L1 activity in a patient identified as having not progressed following definitive chemoradiation therapy.

In one aspect, the disclosure provides a method of extending progression-free survival (PFS) in a patient with, unresectable non-small-cell lung cancer (NSCLC), the method comprising treating the patient with a human anti-PD-L1 antibody, wherein the patient is at a stage III patient who has not progressed following definitive chemoradiation therapy.

In another aspect, the method provides an increase in PFS of at least five months relative to placebo. In further aspects, the method provides an increase in PIPS of at least 13 months relative to placebo.

In one aspect, the disclosure provides a method of increasing the overall response rate (ORR) in a patient with, unresectable NSCLC, the method comprising treating the patient with a human anti-PD-L1 antibody, wherein the patient is at a stage III patient who has not progressed following definitive chemoradiation therapy.

In some aspects, the method provides an increase in ORR of at 12% relative to placebo.

In another aspect, the disclosure provides a method of increasing the time to death or metastasis (TTDM) in a patient with, unresectable NSCLC, the method comprising treating the patient with a human anti-PD-L1 antibody, wherein the patient is at a stage III patient who has not progressed following definitive chemoradiation therapy.

In a further aspect, the disclosure provides a method of lowering incidences of metastasis in a patient with unresectable NSCLC, the method comprising treating the patient with a human anti-PD-L1 antibody, wherein the patient is at a stage III patient who has not progressed following definitive chemoradiation therapy. In some aspects, the lower incidence of metastasis may be in lymph nodes, brain, lung, liver, adrenal gland, bone, abdomen, biliary tract, breast, chest, kidney, ovary, pancreas, pericardium, peritoneal fluid, peritoneum, retroperitoneum, skin, spleen, and/or uterus. In some aspects, the lower incidence of metastasis may be in lymph nodes, brain, lung, liver, adrenal gland, and/or bone.

In a related aspect, the disclosure provides a method of lowering incidences of brain metastasis in a patient with, unresectable NSCLC, the method comprising treating the patient with a human anti-PD-L1 antibody, wherein the patient is at a stage III patient who has not progressed following definitive chemoradiation therapy.

In some aspects, the method provides lowered incidence of metastasis or lowered incidence of metastasis of at least about 20% to about 50% relative to placebo. In some aspects the method provides a decrease in the incidences of metastasis or of brain metastasis by at least five months versus placebo.

In another aspect, the disclosure provides a method treating a patient with stage III locally advanced, unresectable NSCLC, the method comprising treating the patient with a human anti-PD-L1 antibody, wherein the patient has not progressed following definitive chemoradiation therapy.

In certain aspects of any of the above aspects, the chemoradiation therapy comprises a platinum-based therapeutic agent. In some aspects, the platinum-based therapeutic agent may be selected from cisplatin or carboplatin, or a combination of cisplatin and carboplatin.

In some aspects of any of the above aspects, the human anti-PD-L1 antibody comprises durvalumab (IMFINZI®), avelumab (BAVENCIO®), or atezolizumab (TECENTRIQ®). In further aspects the human anti-PD-L1 antibody comprises durvalumab. In aspects, the human anti-PD-L1 antibody comprises a light chain variable domain comprising the amino acid sequence of SEQ ID NO: 1 and a heavy chain variable domain comprising the amino acid sequence of SEQ ID NO: 2. In further aspects, the human anti-PD-L1 antibody comprises heavy and light chain variable region CDRs sequences, wherein the VH CDR1 has the amino acid sequence of SEQ ID NO: 3; the VH CDR2 has the amino acid sequence of SEQ ID NO: 4; the VH CDR3 has the amino acid sequence of SEQ ID NO: 5; the VL CDR1 has the amino acid sequence of SEQ ID NO: 6; the VL CDR2 has the amino acid sequence of SEQ ID NO: 7; and the VL CDR3 has the amino acid sequence of SEQ ID NO: 8.

In aspects of any of the above aspects, the treatment comprises administering the human anti-PD-L1 antibody intravenously once every 2 weeks, at a dosage of 10 mg/kg.

In aspects of any of the above aspects, the patient may express genes (i.e., have a phenotype) associated with therapeutic response to a therapy comprising a human anti-PD-L1 antibody. In some aspects, the patient is PD-L1 (+). In other aspects, the patient is PD-L1 (−). In some aspects, the patient is EGFR mutation (+). In other aspects, the patient is EGFR mutation (−) or wild type. In some aspects, the patient may express any combination of PD-L1 and EGFR mutation phenotypes.

In another aspect, the disclosure provides a method of extending progression-free survival (PFS) in a patient with, unresectable NSCLC, the method comprising treating the patient with a human anti-PD-1 antibody, wherein the patient is at a stage III patient who has not progressed following definitive chemoradiation therapy.

In aspects of this aspect, the chemoradiation therapy comprises a platinum-based therapeutic agent. In some aspects, the platinum-based therapeutic agent may be selected from cisplatin or carboplatin, or a combination of cisplatin and carboplatin.

In some aspects of this aspect, the human anti-PD1 antibody comprises nivolumab (OPDIVO®) or pembrolizumab (KEYTRUDA®).

In some aspects, the method provides an increase in PFS of at least five months relative to placebo. In some aspects, the method provides an increase in PFS of at least thirteen months relative to placebo.

In aspects of this aspect, the patient may express genes (i.e., have a phenotype) associated with therapeutic response to a therapy comprising a human anti-PD-1 antibody. In some aspects, the patient is PD-L1 (+). In other aspects, the patient is PD-L1 (−). In some aspects, the patient is EGFR mutation (+). In other aspects, the patient is EGFR mutation (−) or wild type. In some aspects, the patient may express any combination of PD-L1 and EGER mutation phenotypes.

In a further aspect, the disclosure provides a method of lowering incidences of metastasis in a patient with, unresectable NSCLC, the method comprising treating the patient with a human anti-PD-1 antibody, wherein the patient is at a stage III patient who has not progressed following definitive chemoradiation therapy. In some aspects, the lower incidence of metastasis may be in lymph nodes, brain, lung, liver, adrenal gland, bone, abdomen, biliary tract, breast, chest, kidney, ovary, pancreas, pericardium, peritoneal fluid, peritoneum, retroperitoneum, skin, spleen, and/or uterus. In some aspects, the lower incidence of metastasis may be in lymph nodes, brain, lung, liver, adrenal gland, and/or bone.

In a related aspect, the disclosure provides a method of lowering incidences of brain metastasis in a patient with, unresectable NSCLC, the method comprising treating the patient with a human anti-PD-1 antibody, wherein the patient is at a stage III patient who has not progressed following definitive chemoradiation therapy.

In some aspects, the method provides lowered incidence of metastasis or lowered incidence of metastasis of at least about 20% to about 50% relative to placebo. In some aspects the method provides a decrease in the incidences of metastasis or of brain metastasis by at least five months versus placebo.

In aspects of these related aspects, the patient may express genes (i.e., have a phenotype) associated with therapeutic response to a therapy comprising a human anti-PD-L1 antibody. In some aspects, the patient is PD-L1 (+). In other aspects, the patient is PD-L1 (−). In some aspects, the patient is EGFR mutation (+). In other aspects, the patient is EGFR mutation (−) or wild type. In some aspects, the patient may express any combination of PD-L1 and EGFR mutation phenotypes.

In various aspects of the above aspects, treatment may comprise administration of at least about 10 mg/kg durvalumab, or an antigen-binding fragment thereof. In some aspects, the administration is repeated about every 14 days, for up to 52 weeks.

Other features, aspects, aspects, and advantages of provided by the disclosure will be apparent from the detailed description that follows.

### DETAILED DESCRIPTION

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In this disclosure, “comprises,” “comprising,” “containing” and “having” and the like can have the meaning ascribed to them in U.S. patent law and can mean “ includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art aspects.

Unless specifically stated or obvious from context, as used herein, the term “or” is understood to be inclusive. Unless specifically stated or obvious from context, as used herein, the terms “a” “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an aspect for a variable or aspect herein includes that aspect as any single aspect or in combination with any other aspects or portions thereof.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “anti-PD-L1 antibody” is meant an antibody or antigen binding fragment thereof that selectively binds a PD-L1 polypeptide. Exemplary anti-PD-L1 antibodies are described for example at U.S. Pat. Nos. 8,779,108 and 9,493,565, which are herein incorporated by reference. In some aspects durvalumab, avelumab, or atezolizumab durvalumab is an exemplary PD-L1 antibody. In further aspects, durvalumab is an exemplary PD-L1 antibody.

By “anti-PD-1 antibody” is meant an antibody or antigen binding fragment thereof that selectively binds a PD-1 polypeptide. In some aspects nivolumab or pembrolizumab is an exemplary PD-1 antibody.

A “complete response” (CR) refers to the disappearance of all lesions, whether measurable or not, and no new lesions. Confirmation can be obtained using a repeat, consecutive assessment no less than four weeks from e date of first documentation. New, non-measurable lesions preclude CR.

A “partial response” (PR) refers to a decrease in tumor burden≥50% relative to baseline. Confirmation can be obtained using a consecutive repeat assessment at least 4 weeks from the date of first documentation.

“Progressive disease” (PD) refers to an increase in tumor burden≥25% relative to the minimum recorded (nadir). Confirmation can be obtained by a consecutive repeat assessment at least 4 weeks from the date of first documentation. New, non-measurable lesions do not define PD.

“Stable disease” (SD) refers to not meeting the criteria for CR, PR, or PD. SD indicates a decrease in tumor burden of 50% relative to baseline cannot be established and a 25% increase compared to nadir cannot be established.

Non-small cell lung cancer (NSCLC) can refer to any of the three main subtypes of NSCLC: squamous cell carcinoma, adenocarcinoma, and large cell (undifferentiated) carcinoma. Other subtypes include adenosquamous carcinoma and sarcomatoid carcinoma.

As referred to herein, “PD-L1” may refer to polypeptide or polynucleotide sequences, or fragments thereof, having at least about 85%, 95% or 100% sequence identity to PD-L1 sequences. PD-L1 is also referred to in the art as B7-H1. In some aspects, the PD-L1 polypeptide, or fragment thereof, has at least about 85%, 95% or 100% sequence identity to NCBI Accession No. NP_001254635, and has PD-1 and CD80 binding activity.

| PD-L1 polypeptide sequence | |
|---|---|
| NCBI ACCESSION NO. NP_001254635 | |
| 1 | mrifavfifm tywhllnapy nkinqrilvv dpvtsehelt cqaegypkae viwtssdhqv |
| 61 | lsgkttttns kreeklfnvt stlrintttn eifyctfrrl dpeenhtael vipelplahp |
| 121 | pnerthlvil gaillclgva ltfifrlrkg rmmdvkkcgi qdtnskkqsd thleet |

In some aspects a “PD-L1 nucleic acid molecule” comprises a polynucleotide encoding a PD-L1 polypeptide. An exemplary PD-L1 nucleic acid molecule sequence is provided at NCBI Accession No. NM_001267706.

| PD-L1 nucleic acid sequence | |
|---|---|
| NCBI ACCESSION NO. NM_001267706 mRNA | |
| 1 | ggcgcaacgc tgagcagctg gcgcgtcccg cgcggcccca gttctgcgca gcttcccgag |
| 61 | gctccgcacc agccgcgctt ctgtccgcct gcagggcatt ccagaaagat gaggatattt |
| 121 | gctgtcttta tattcatgac ctactggcat ttgctgaacg ccccatacaa caaaatcaac |
| 181 | caaagaattt tggttgtgga tccagtcacc tctgaacatg aactgacatg tcaggctgag |
| 241 | ggctacccca aggccgaagt catctggaca agcagtgacc atcaagtcct gagtggtaag |
| 301 | accaccacca ccaattccaa gagagaggag aagcttttca atgtgaccag cacactgaga |
| 361 | atcaacacaa caactaatga gattttctac tgcactttta ggagattaga tcctgaggaa |
| 421 | aaccatacag ctaaattggt catcccagaa ctacctctgg cacatcctcc aaatgaaagg |
| 481 | actcacttgg taattctggg agccatctta ttatgccttg gtgtagcact gacattcatc |
| 541 | ttccgtttaa gaaaagggag aatgatggat gtgaaaaaat gtggcatcca agatacaaac |
| 601 | tcaaagaagc aaagtgatac acatttggag gagacgtaat ccagcattgg aacttccgat |
| 661 | cttcaagcag ggattctcaa cctgtggttt aggggttcat cggggctgag cgtgacaaga |
| 721 | ggaaggaatg ggcccgtggg atgcaggcaa cgtgggactt aaaaggccca agcactgaaa |
| 781 | atggaacctg gcgaaagcag aggaggagaa tgaagaaaga tggagtcaaa cagggagcct |
| 841 | ggagggagac cttgatactt tcaaatgcct gaggggctca tcgacgcctg tgacagggag |
| 901 | aaaggatact tctgaacaag gagcctccaa gcaaatcatc cattgctcat cctaggaaga |
| 961 | cgggttgaga atccctaatt tgagggtcag ttcctgcaga agtgcccttt gcctccactc |
| 1021 | aatgcctcaa tttgttttct gcatgactga gagtctcagt gttggaacgg gacagtattt |
| 1081 | atgtatgagt ttttcctatt tattttgagt ctgtgaggtc ttcttgtcat gtgagtgtgg |
| 1141 | ttgtgaatga tttcttttga agatatattg tagtagatgt tacaattttg tcgccaaact |
| 1201 | aaacttgctg cttaatgatt tgctcacatc tagtaaaaca tggagtattt gtaaggtgct |
| 1261 | tggtctcctc tataactaca agtatacatt ggaagcataa aaatcaaacc gttggttgca |
| 1321 | taggatgtca cctttattta acccattaat actctggttg acctaatctt attctcagac |
| 1381 | ctcaagtgtc tgtgcagtat ctgttccatt taaatatcag ctttacaatt atgtggtagc |
| 1441 | ctacacacat aatctcattt catcgctgta accaccctgt tgtgataacc actattattt |
| 1501 | tacccatcgt acagctgagg aagcaaacag attaagtaac ttgcccaaac cagtaaatag |
| 1561 | cagacctcag actgccaccc actgtccttt tataatacaa tttacagcta tattttactt |
| 1621 | taagcaattc ttttattcaa aaaccattta ttaagtgccc ttgcaatatc aatcgctgtg |
| 1681 | ccaggcattg aatctacaga tgtgagcaag acaaagtacc tgtcctcaag gagctcatag |
| 1741 | tataatgagg agattaacaa gaaaatgtat tattacaatt tagtccagtg tcatagcata |
| 1801 | aggataatgc gaggggaaaa cccgagcagt gttgccaaga gaaggaaata ggccaatgtg |
| 1861 | gtctgggacg gttggatata cttaaacatc ttaataatca gagtaatttt catttacaaa |
| 1921 | gagaggtcgg tacttaaaat aaccctgaaa aataacactg gaattccttt tctagcatta |
| 1981 | tatttattcc tgatttgcct ttgccatata atctaatgct tgtttatata gtgtctggta |
| 2041 | ttgtttaaca gttctgtctt ttctatttaa atgccactaa attttaaatt catacctttc |
| 2101 | catgattcaa aattcaaaag atcccatggg agatggttgg aaaatctcca cttcatcctc |
| 2161 | caagccattc aagtttcctt tccagaagca actgctactg cctttcattc atatgttctt |
| 2221 | ctaaagatag tctacatttg gaaatgtatg ttaaaagcac gtatttttaa aatttttttc |
| 2281 | ctaaatagta acacattgta tgtctgctgt gtactttgct atttttattt attttagtgt |
| 2341 | ttcttatata gcagatggaa tgaatttgaa gttcccaggg ctgaggatcc atgccttctt |
| 2401 | tgtttctaag ttatctttcc catagctttt cattatcttt catatgatcc agtatatgtt |
| 2461 | aaatatgtcc tacataLaca tttagacaac caccatttgt taagtatttg ctctaggaca |
| 2521 | gagtttggat ttgtttatgt ttgctcaaaa ggagacccat gggctctcca gggtgcactg |
| 2581 | agtcaatcta gtcctaaaaa gcaatcttat tattaactct gtatgacaga atcatgtctg |
| 2641 | gaacttttgt tttctgcttt ctgtcaagta taaacttcac tttgatgcta tacttgcaaa |
| 2701 | abcacatttt ctttctggaa attccggcag tgtaccttga ctgctagcta ccctgtgcca |
| 2761 | gaaaagcctc attcgttgtg cttgaaccct tgaatgccac cagctgtcat cactacacag |
| 2821 | ccctcctaag aggcttcctg gaggtttcga gattcagatg ccctgggaga tcccagagtt |
| 2881 | tcctttccct cttggccata ttctggtgtc aatgacaagg agtaccttgg ctttgccaca |
| 2941 | tgtcaaggct gaagaaacag tgtccccaac agagctccCt gtgttatctg tttgtacatg |
| 3001 | tgcatttgta cagtaattgg tgtgacagtg ttctttgtgt gaattacagg caagaattgt |
| 3061 | ggctgagcaa ggcacatagt ctactcagtc tattcctaag tcctaactcc tccttgtggt |
| 3121 | gttggatttg taaggcactt tatccctttt gtctcatgtt tcatcgtaaa tggcataggc |
| 3181 | agagatgata cctaattctg catttgattg tcactttttg tacctgcatt aatttaataa |
| 3241 | aatattctta tttattttgt tacttggtac accagcatgt ccattttctt gtttattttg |
| 3301 | tgtttaataa aatgttcagt ttaacatccc agtgaagaaa gttaaaaaa |

Programmed Death-1 (“PD-1”) is an approximately 31 kD type I membrane protein member of the extended CD28/CTLA4 family of T cell regulators (see, Ishida, Y. et al. (1992) Induced Expression Of PD-1, A Novel Member Of The Immunoglobulin Gene Superfamily, Upon Programmed Cell Death,” EMBO J. 11 :3887-3895.

PD-1 is expressed on activated T cells, B cells, and monocytes (Agata, Y. et al. (1996) “Expression Of The PD-1 Antigen On The Surface Of Stimulated Mouse T And B Lymphocytes,” Int. Immunol. 8(5):765-772; Yamazaki, T. et al. (2002) “Expression Of Programmed Death 1 Ligands By Murine T Cells And APC,” J. Immunol. 169:5538-5545) and at low levels in natural killer (NK) T cells (Nishimura, H. et al. (2000) “Facilitation of Beta Selection and Modification of Positive Selection in the Thymus of PD-1-Deficient Mice,” J. Exp. Med. 191: 891-898; Martin-Orozco, N. et al. (2007) “Inhibitory Costimulation And Anti-Tumor Immunity,” Semin. Cancer Biol. 17(4):288-298). PD-1 is a receptor responsible for down-regulation of the immune system following activation by binding of PDL-1 or PDL-2 (Martin-Orozco, N. et al. (2007) “Inhibitory Costimulation And Anti-Tumor Immunity,” Semin. Cancer Biol. 17(4):288-298) and functions as a cell death inducer (Ishida, Y. et al. (1992) “Induced Expression of PD-1, A Novel Member of the Immunoglobulin Gene Superfamily, Upon Programmed Cell Death,” EMBO J. 11: 3887-3895; Subudhi, S. K. et al. (2005) “The Balance Of Immune Responses: Costimulation Verse Coinhibition,” J. Molec. Med. 83: 193-202) (Lazar-Molnar, E. et al. (2008) “Crystal Structure of the Complex Between Programmed Death-1 (PD-1) And Its Ligand PD-L2,” Proc. Natl. Acad. Sci. (USA) 105(30): 10483-10488). This process is exploited in many tumours via the over-expression of PD-L1, leading to a suppressed immune response.

PD-1 is a well-validated target for immune mediated therapy in oncology, with positive clinical trials in the treatment of melanoma and non-small cell lung cancers (NSCLC), among others. Antagonistic inhibition of the PD-1/PDL-1 interaction increases T cell activation, enhancing recognition and elimination of tumour cells by the host immune system. The use of anti-PD-1 antibodies to treat infections and tumors and up-modulate an adaptive immune response has been proposed.

The term “antibody,” as used in this disclosure, refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)₂, Fv, scFv, Fd, dAb, and other antibody fragments that retain antigen-binding function, i.e., the ability to bind PD-L1 specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible, for specific interactions with the antigen-binding domain is referred to as “epitope” or “antigenic determinant.” An antigen-binding domain typically comprises an antibody light chain variable region (V_{L}) and an antibody heavy chain variable region (V_{H}), however, it does not necessarily have to comprise both. For example, a so-called Fd antibody fragment consists only of a V_{H} domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab′)2 fragment in which the two arms of the antibody molecule remain linked and comprise two-antigen binding sites. The F(ab′)2 fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

The term “mAb” refers to monoclonal antibody. Antibodies of the invention comprise without limitation whole native antibodies, bispecific antibodies; chimeric antibodies; Fab, Fab′, single chain V region fragments (scFv), fusion polypeptides, and unconventional antibodies.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences.

By “specifically binds” is meant a compound (e.g., antibody) that recognizes and binds a molecule (e.g., polypeptide), but which does not substantially recognize and bind other molecules in a sample, for example, a biological sample. For example, two molecules that specifically bind form a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant. K_{A} is higher than 10⁶ M⁻¹, or more preferably higher than 10⁸ M⁻¹. If necessary, non-specific binding can be reduced without substantially affecting specific binding by varying the binding conditions. The appropriate binding conditions such as concentration of antibodies, ionic strength of the solution, temperature, time allowed for binding, concentration of a blocking agent (e.g., serum albumin, milk casein), etc., may be optimized by a skilled artisan using routine techniques.

As generally used herein, the terms “treat,” treating,” “treatment,” and the like refer to reducing, ameliorating, or slowing the progression of a disorder or disease and/or symptoms associated with a disorder or disease. It will be appreciated that, although not precluded, treating a disorder, disease, or condition does not require that the disorder, disease, or condition or associated symptoms be completely eliminated. In particular aspects and aspects relating to NSCLC, “treat,” treating,” “treatment,” can refer to achieving any one or combination of primary or secondary clinical endpoints.

### BRIEF DESCRIPTION OF THE DRAWINGS

FIG. 1 provides statistical analysis showing Progression-free Survival (PFS) in the Intention-to-Treat Population by Blinded Independent Central Review (BICR). Kaplan-Meier curves for PFS (defined by Response Evaluation Criteria In Solid Tumors (RECIST v1.1); and assessed by BICR) in patients receiving durvalumab or placebo. The total number of events/total number of patients are 214/476 (durvalumab) and 157/237 (placebo); median PFS in months 16.8 (13.0-18.1, 95% CI (durvalumab)) and 5.6 (4.6-7.8, 95% CI (placebo)); 12-month PFS rate 55.9% (51.0-60.4%, 95% CI (durvalumab)) and 44.2% (37.7-50.5%, 95% CI (placebo)); and 18-month PFS rate 35.3% (29.0-41.7% 95% CI (durvalumab)) and 27.0% (19.9-34.5%, 95% CI (placebo)). Symbols indicate censored observations. The intention-to-treat population included all patients who underwent randomization.

FIG. 2 depicts PFS (defined by RECIST v.1.1) subgroup analysis of prognostic factors in the intention-to-treat population (assessed by BICR). Hazard ratio and 95% CI is not calculated if the subgroup level had less than 20 events. CR is complete response; EGFR is epidermal growth factor receptor; PD-L1 is programmed cell death ligand-1; PR is partial response; SD is stable disease; WHO is World Health Organization.

FIG. 3 depicts a CONSORT flow diagram of the data obtained in the clinical trial. ^{†}Four patients (3 in the durvalumab group and 1 in the placebo group) were randomized but did not receive treatment because of patient decision (n=2), neutropenia (n=1), and worsening chronic obstructive pulmonary disease (n=1). ^{‡}Patients who completed 12 months of treatment reported the maximum cycle of immunotherapy reached on the electronic case report form (eCRF). ^{¶}Two patients randomized to placebo received one dose of durvalumab and were included in the safety analysis set.

FIG. 4 depicts PFS (defined by RECIST v.1.1) subgroup analysis of additional factors in the intention-to-treat population (assessed by BICR). Hazard ratio and 95% CI is not calculated if the subgroup level has less than 20 events.

FIG. 5 depicts incidence of new lesions by BICR (ITT), can include more than one new lesion site. Other includes lesions in: abdominal wall, biliary tract, breast, chest wall, kidney, ovary, pancreas, pericardium, peritoneal fluid, peritoneum, retroperitoneum, skin, spleen, uterus and other (unspecified).

FIG. 6 depicts statistical analysis of time to death or distant metastasis (TDDM) in the intention-to-treat population. The probability of death or distant metastasis is associated with a median TDDM of 23.2 months (23.2-NR, 95% CI) for durvalumab and 14.6 months (10.6-8.6, 95% CI) for placebo. The calculated Hazard ratio is 0.52 (95% CI, 0.39-0.69).

FIG. 7 depicts statistical analysis of duration of response in the intention-to-treat population, plotted as proportion of patients remaining in response at close of clinical evaluation as a function of time (months). The median duration of response (DoR), in months, for durvalumab was ‘not reported’ while placebo exhibited a median DoR of 13.8 months (6.0-NR, 95% CI).

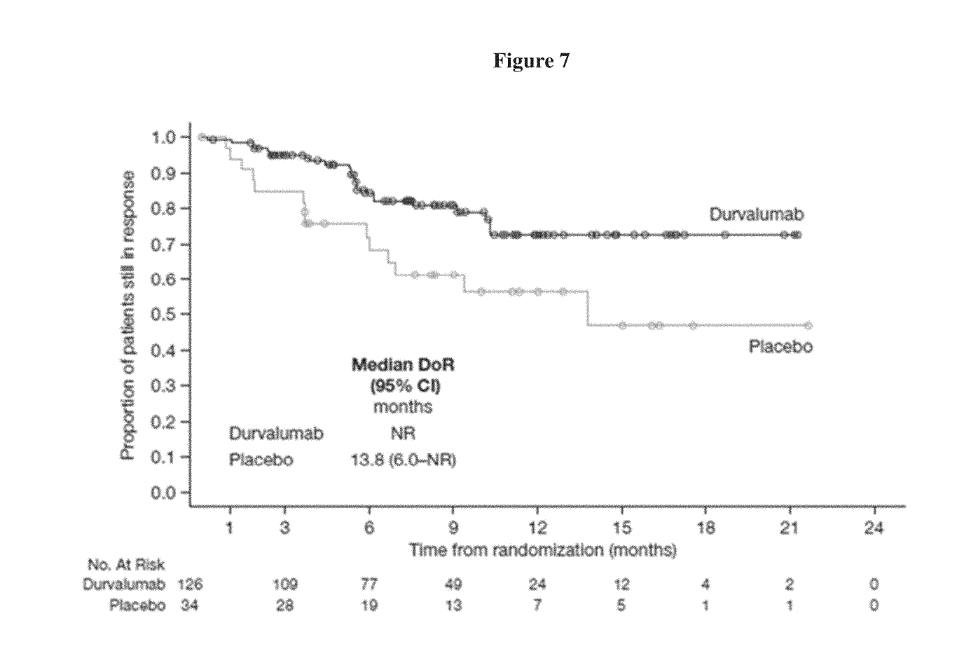

### SEQUENCES

Durvalumab light chain variable region amino acid sequence is provided as SEQ ID NO: 1

Durvalumab heavy chain variable region amino acid sequence is provided as SEQ ID NO: 2.

Durvalumab heavy chain variable region amino acid sequence of CDR1, CDR2, and CDR3 are provided as SEQ ID NO: 3 (CDR1), SEQ ID NO: 4 (CDR2), and SEQ ID NO: 5 (CDR3).

Durvalumab light chain variable region amino acid sequence of CDR1, CDR2, and CDR3 are provided as SEQ ID NO: 6 (CDR1), SEQ ID NO: 7 (CDR2), and SEQ ID NO: 8 (CDR3).

The disclosure relates to methods of treating patients who have unresectable, late stage non-small-cell lung cancer (NSCLC) who have not progressed following definitive chemoradiation therapy, comprising administering to the patient a human anti-PD-L1 antibody. In particular, the data derived from the clinical results disclosed herein provide for improved treatment methods and substantially redefine the existing standard of care for treatment of unresectable, late stage (e.g., stage III) non-small-cell lung cancer (NSCLC) in patients who have not progressed following definitive chemoradiation therapy. The disclosed methods of treatment can provide for substantial improvement in a patient's progression-free survival (PFS), overall response rate (ORR), time to death or metastasis (TTDM), duration of response (DoR), and/or lower the incidences of metastatic spread of the NSCLC in the patient. The data supports new standard of care methods for the treatment of locally advanced, unresectable, late stage non-small-cell lung cancer (NSCLC) who have not progressed following definitive chemoradiation therapy.

Thus, in the various aspects below, the disclosed methods provide for treating a patient with, locally advanced, unresectable NSCLC, and who has not progressed following definitive chemoradiation therapy, where the treatment can extend progression-free survival (PFS); increase the overall response rate (ORR); increase the time to death or metastasis (TTDM); lower incidences of metastasis; lower incidences of brain metastasis; increase overall survival (OS); increase duration of response (DoR); and/or increase the proportion of patients alive and progression free (APF).

In one aspect, the disclosure provides a method of extending progression-free survival (PFS) in a patient with, unresectable non-small-cell lung cancer (NSCLC), the method comprising treating the patient with a human anti-PD-L1 antibody, wherein the patient is at a stage III patient who has not progressed following definitive chemoradiation therapy.

In one aspect, the disclosure provides a method of increasing the overall response rate (ORR) in a patient with, unresectable NSCLC, the method comprising treating the patient with a human anti-PD-L1 antibody, wherein the patient is at a stage III patient who has not progressed following definitive chemoradiation therapy.

In another aspect, the disclosure provides a method of increasing the time to death or metastasis (TTDM) in a patient with, unresectable NSCLC, the method comprising treating the patient with a human anti-PD-L1 antibody, wherein the patient is at a stage III patient who has not progressed following definitive chemoradiation therapy.

In a further aspect, the disclosure provides a method of lowering incidences of metastasis in a patient with, unresectable NSCLC, the method comprising treating the patient with a human anti-PD-L1 antibody, wherein the patient is at a stage III patient who has not progressed following definitive chemoradiation therapy.

In a related aspect, the disclosure provides a method of lowering incidences of brain metastasis in a patient with, unresectable NSCLC, the method comprising treating the patient with a human anti-PD-L1 antibody, wherein the patient is at a stage III patient who has not progressed following definitive chemoradiation therapy.

In another aspect, the disclosure provides a method treating a patient with stage III locally advanced, unresectable NSCLC, the method comprising treating the patient with a human anti-PD-L1 antibody, wherein the patient has not progressed following definitive chemoradiation therapy.

In another aspect, the disclosure provides a method of extending progression-free survival (PFS) in a patient with, unresectable NSCLC, the method comprising treating the patient with a human anti-PD-1 antibody, wherein the patient is at a stage III patient who has not progressed following definitive chemoradiation therapy.

In a further aspect, the disclosure provides a method of lowering incidences of metastasis in a patient with, unresectable NSCLC, the method comprising treating the patient with a human anti-PD-1 antibody, wherein the patient is at a stage III patient who has not progressed following definitive chemoradiation therapy.

In an aspect, the disclosure provides a method of lowering incidences of brain metastasis in a patient with, unresectable NSCLC, the method comprising treating the patient with a human anti-PD-1 antibody, wherein the patient is at a stage III patient who has not progressed following definitive chemoradiation therapy.

In aspects of these aspects, the chemoradiation therapy comprises a platinum-based therapeutic agent.

In some aspects of the above aspects, the lower incidence of metastasis may be in lymph nodes, brain, lung, liver, adrenal gland, bone, abdomen, biliary tract, breast, chest, kidney, ovary, pancreas, pericardium, peritoneal fluid, peritoneum, retroperitoneum, skin, spleen, and/or uterus. In some aspects, the lower incidence of metastasis may be in lymph nodes, brain, lung, liver, adrenal gland, and/or bone.

In some aspects of the above aspects, the human anti-PD-L1 antibody comprises durvalumab (IMFINZI®), avelumab (BAVENCIO®), or atezolizumab (TECENTRIQ®). In further aspects the human anti-PD-L1 antibody comprises durvalumab. In aspects, the human anti-PD-L1 antibody comprises a light chain variable domain comprising the amino acid sequence of SEQ ID NO: 1 and a heavy chain variable domain comprising the amino acid sequence of SEQ ID NO: 2. In further aspects, the human anti-PD-L1 antibody comprises heavy and light chain variable region CDRs sequences, wherein the VH CDR1 has the amino acid sequence of SEQ ID NO: 3; the VH CDR2 has the amino acid sequence of SEQ ID NO: 4; the VH CDR3 has the amino acid sequence of SEQ ID NO: 5; the VL CDR1 has the amino acid sequence of SEQ ID NO: 6; the VL CDR2 has the amino acid sequence of SEQ ID NO: 7; and the VL CDR3 has the amino acid sequence of SEQ ID NO: 8.

In aspects of the above aspects, the treatment comprises administering the human anti-PD-L1 antibody intravenously once every 2 weeks, at a dosage of 10 mg/kg.

In aspects of the above aspects, the method provides an increase in PFS of at least five months relative to placebo. In further aspects, the method provides an increase in PFS of at least 13 months relative to placebo.

In some aspects of the above aspects, the method provides an increase in ORR of at 12% relative to placebo.

In some aspects of the above aspects, the method provides an increase in TDDM of at least four months versus placebo.

In some aspects of the above aspects, the method provides lowered incidence of metastasis or lowered incidence of metastasis of at least about 20% to about 50% relative to placebo. In some aspects the method provides a decrease in the incidences of metastasis or of brain metastasis by at least five months versus placebo.

In some aspects of the above aspects, the human anti-PD1 antibody comprises nivolumab (OPDIVO®) or pembrolizumab (KEYTRUDA®).

In certain aspects the patient is administered one or more doses of an anti-PD-1 antibody, wherein the dose is a fixed dose of 200 mg.

In certain aspects the patient is administered one or more doses of an anti-PD-1 antibody, wherein the dose is a fixed dose of 240 mg.

In certain aspects the patient is administered one or more doses of an anti-PD-1 antibody, wherein the dose is a fixed dose of 480 mg.

In some aspects, an anti-PD-1 antibody or an antigen-binding fragment thereof is administered every two weeks.

In some aspects, an anti-PD-1 antibody or an antigen-binding fragment thereof is administered every three weeks.

In some aspects, an anti-PD-1 antibody or an antigen-binding fragment thereof is administered every four weeks.

In aspects of the above aspects, the patient may express genes (i.e., have a phenotype) associated with therapeutic response to a therapy comprising a human anti-PD-L1 antibody. In some aspects, the patient is PD-L1 (+). In other aspects, the patient is PD-L1 (−). A sample was determined to be “PD-L1 positive” if the sample contained 25% or more tumor cells with PDL1 membrane staining. A cutoff and scoring algorithm has been previously determined for durvalumab (Study CP1108; ClinicalTrials.gov number NCT01693562).

In some aspects, the patient is EGFR mutation (+). In other aspects, the patient is EGFR mutation (−) or wild type. In some aspects, the patient may express any combination of PD-L1 and EGFR mutation phenotypes.

In various aspects of the above aspects, treatment may comprise administration of at least about 10 mg/kg durvalumab, or an antigen-binding fragment thereof. In some aspects, the administration is repeated about every 14 days, for up to 52 weeks.

Overall Survival (OS) relates to the time period beginning on the date of treatment until death due to any cause. OS may refer to overall survival within a period of time such as, for example, 12 months, 18 months, 24 months, and the like. Such periods of time can be identified, for example, as “OS24” which refers to the number (%) of patients who are alive at 24 months after treatment onset per the Kaplan-Meier estimate of overall survival at 24 months.

Progression-Free Survival (PFS) relates to the time period beginning on the date of treatment until the date of objective disease progression (RECIST 1.1) or death (by any cause in the absence of progression). In some aspects the methods provide for increase in PFS. In some aspects the methods provide for PFS of at least 9 months to at least about 24 months (e.g., at least 9, 10, 11, 12, 13, 14, 15, 16, 17, 18, 19, 20, 21, 22, 23, 24, or more months, and up to about 5 years).

Duration of Response (DoR) refers to the time from the date for first documented response of Complete Response (CR) or Partial Response (PR) until the first documented response of progression per RECIST 1.1 or death in the absence of progression. In aspects the methods provide for an increase in DoR of at least about 9 months to at least about 36 months.

Objective Response Rate (ORR) refers to the number (%) of patients with at least one visit response of Complete Response (CR) or partial response (PR) per RECIST 1.1. In aspects the methods provide for an increase in DoR of at least about 9 months to at least about 36 months.

Proportion of patients alive and progression free (APF) refers to the number (%) of patients who are alive and progression free per RECIST 1.1. APF may refer to a period of time such as, for example, 12 months, 18 months, 24 months, and the like. Such periods of time can be identified, for example, as at APF12 identifying the number of patients alive and progression free at 12 months after treatment onset per Kaplan-Meier estimate of progression free survival at 12 months.

Time to death or distant metastasis (TTDM) refers any new lesion that is outside of the radiation field. In some aspects the methods provide for increase in TTDM. In some aspects the methods provide for TTDM of at least 9 months to at least about 24 months (e.g., at least 9, 10, 11, 12, 13, 14, 15, 16, 17, 18, 19, 20, 21, 22, 23, 24, or more months, and up to about 5 years).

In aspects, the methods are administered to a patient who has not progressed following definitive, concurrent chemoradiation therapy. In some aspects, the concurrent chemoradiation therapy comprises any accepted standard first-line treatments for patients with advanced NSCLC. In certain aspects, standard first-line treatments may include chemotherapy, radiation therapy, or both (chemoradiation therapy). In some aspects the therapy can comprise one or more platinum-based chemotherapeutic agents. In some aspects, the one or more platinum-based chemotherapeutic agents can be selected from carboplatin, cisplatin, oxaliplatin, or combinations thereof. As further described herein the platinum-based therapy can comprise singlet or doublet regimens such as, for example, administering cisplatin or carboplatin with another anticancer agent such as paclitaxel, docetaxel, etoposide, gemcitabine, vinorelbine, and the like.

The disclosed methods comprise administration of a therapeutic; (e.g., a human anti-PD-L1 antibody or a human anti-PD-1 antibody) following a prior therapeutic regimen that failed to achieve one or more clinical endpoints. In certain aspects the method is performed following definitive chemoradiation therapy that comprises a platinum-based drug as discussed above. In some aspects, the methods is performed following 1, 2, or more rounds of definitive chemoradiation therapy that comprises a platinum-based drug, and which do not inhibit progression of the NSCLC. In some aspects of the methods of treatment provided herein, administration of the human anti-PD-L1 antibody or a human anti-PD-1 antibody can begin once a determination that the NSCLC was not responsive to the prior therapeutic regimen. In some aspects, the patient may be treated within 1 to about 42 days (e.g., 1, 2, 3, 4, 5, 6, 7, 14, 21, 28, 35, or 42 days or more) after the patient had received chemoradiation therapy.

As described herein and illustrated by the examples, the methods provide for the treatment of locally advanced, unresectable NSCLC. In some aspects, an “unresectable” cancer includes cancer that cannot be removed completely through surgery for at least one of several medical reasons. Reasons why a cancer may be unresectable include, for example, tumor size (e.g., too large to safely remove and/or may require extensive removal of a part of an essential organ), tumor location (e.g., tumor physically intertwined vital structures such as blood vessels or nerves), tumor metastasis where removal of the tumor will not be effective to control all of the cancer, or other medical conditions that heighten risk of surgery to an unacceptable level (e.g., heart disease, lung disease, diabetes). Further, an unresectable NSCLC may not be permanently unresectable after aggressive treatment that may be effective to reduce the size of a tumor to a degree that allows for possible surgical resection. Further, unresectable NSCLC can also refer to NSCLC (or remote metastases) that will not be completely removed by surgery, but which may be partially removed by one or more surgical procedures. Examples include debulking surgery and surgery that removes parts of the lung cancer as well as parts of metastatic lesions.

In certain aspects, the methods disclosed herein can be used on “resectable” cancers.

As mentioned above, and as illustrated herein, the methods treat patients with late-stage (e.g., Stage III) locally advanced, unresectable NSCLC and who have not progressed following definitive chemoradiation therapy. Cancer staging can be performed using any tests that are generally known and accepted in the art. In aspects, the cancer staging can comprise the American Joint Committee on Cancer's (AJCC's) TNM system. Generally the TNM system provides results from various tests and scans in order to determine the size and location of the primary tumor (Tumor, T); whether the cancer has spread to the lymph nodes, and if it has, the location and number of the affected lymph nodes (Node, N); and whether the cancer has spread to other parts of the body, and if it has, the extent and location of the remote cancer (Metastasis, M). While each type of cancer may have its own specific system, the TNM staging system generally uses scaled scoring for each letter.

For Tumor, “T” is associated with a number (e.g., 0 to 4) to describe the general tumor size, location, and whether it intrudes into nearby tissues. Larger or more intrusive tumors are given a higher number and, depending on the cancer, a lowercase letter, such as “a,” “b,” or “m” (for multiple), may be added to provide more detail.

Similarly for Node, “N” is associated with a number (e.g., 0 to 3) to describe whether cancer has been found in the lymph nodes, and can also indicate the number of lymph nodes containing cancer. Larger numbers are assigned when more lymph nodes are involved with cancer.

For Metastasis, “M” indicates whether or not the cancer has spread to other parts of the body and is labeled M0 for no spread, or M1 if it has spread.

The T, N, and M results are combined to determine the stage of cancer, typically one of four stages: stages I (one) to IV (four). Some cancers also have a stage 0 (zero). Stage 0 describes cancer in situ, remaining local to the original tissue without any spread to nearby tissues. This stage of cancer is often highly curable, usually by removing the entire tumor with surgery. Stage or early-stage cancer, is typically used to describe a small cancer or tumor that has not grown deeply into nearby tissues, and has not spread to the lymph nodes or other parts of the body. Stage II and III describe larger cancers or tumors that have grown more deeply into nearby tissue, and that may have also spread to lymph nodes but not metastasized to other tissues. Stage IV describes a cancer that has spread to other organs or parts of the body and often identified as advanced or metastatic cancer.

Staging may include optional analysis of prognostic factors to provide chances of recovery and a recommended therapy. Prognostic factors may include grading the cancer based on appearance of the cancer cells; analysis of tumor marker expression; and analysis of tumor genetics.

A cancer may be restaged using the same initial system in order to determine efficacy of a treatment or obtain more information about a recurrent cancer.

### Staging of NSCLC

NSCLC has 5 stages: a stage 0 (zero) and stages I through IV (1 through 4). Stage 0 NSCLC indicates that the cancer has not grown into nearby tissues or spread outside the lung.

Stage I NSCLC indicates that the cancer is a small tumor that has not spread to any lymph nodes. Stage I is divided into 2 sub-stages based on the size of the tumor: Stage IA tumors are less than 3 centimeters (cm) wide, and Stage IB tumors are more than 3 cm but less than 5 cm wide. Stage I NSCLC may allow for complete surgical removal of the cancer.

Stage II is divided into 2 sub-stages (IIA and IIB). Stage IIA can be either a tumor larger than 5 cm but less than 7 cm wide that has not spread to the nearby lymph nodes, or a small tumor less than 5 cm wide that has spread to the nearby lymph nodes. Stage IIB can describe either a tumor larger than 5 cm but less than 7 cm wide that has spread to the lymph nodes, or a tumor more than 7 cm wide that may or may not have grown into nearby structures in the lung but has not spread to the lymph nodes. While stage II NSCLC may allow for surgical treatment, other therapies are commonly required to treat this stage of NSCLC.

Stage III includes sub-stages IIIA or IIIB. Surgery is difficult or impossible in many stage IIIA cancers and nearly all stage IIIB, because of the spread of the cancer to the lymph nodes or because of its growth into nearby structures in the lung. Surgery in either situation typically requires the partial removal of the cancer.

Stage IV NSCLC is associated with its spread to more than one area in the other lung, the fluid surrounding the lung or the heart, or distant metastasis in the body. NSCLC is more likely to spread to the brain, bones, liver, and adrenal glands. Stage IV NSCLC includes substages IVA (spread within the chest) and IVB (spread outside of the chest). Surgery is rarely successful for most stage III or IV NSCLC and may be impossible to remove if it has spread to the lymph nodes above the collarbone, or to vital structures within the chest (e.g., heart, large blood vessels, or the main pulmonary structures). In certain aspects, a patient disclosed herein is a stage IV NSCLC patient.

Recurrent NSCLC is detected after a course of treatment.

### Anti-PD-L1 Antibodies

Antibodies that specifically bind and inhibit PD-L1 activity (e.g., binding to PD-1 and/or CD80) are useful in the methods disclosed herein.

Durvalumab is an exemplary anti-PD-L1 antibody that is selective for PD-L1 and blocks the binding of PD-L1 to the PD-1 and CD80 receptors. Durvalumab can relieve PD-L1-mediated suppression of human T-cell activation in vitro and inhibits tumor growth in a xenograft model via a T-cell dependent mechanism. Other agents that are useful in the disclosed methods include agents that inhibit PD-L1 and/or PD-1, such as, for example the human anti-PD-L1 antibodies avelumab and atezolizumab, or the human anti-PD-1 antibodies nivolumab and pembrolizumab.

In certain aspects, an antibody that is used in the methods disclosed herein is any agent that disrupts the PD-1/PD-L1 axis.

Information regarding Durvalumab (or fragments thereof) for use in the methods provided herein can be found in U.S. Pat. Nos. 8,779,108 and 9,493,565, the disclosures of which are incorporated herein by reference in its entirety. The fragment crystallizable (Fc) domain of durvalumab contains a triple mutation in the constant domain of the IgG1 heavy chain that reduces binding to the complement component C1q and the Fcγ receptors responsible for mediating antibody-dependent cell-mediated cytotoxicity (ADCC).

Durvalumab and antigen-binding fragments thereof for use in the methods provided herein comprises a heavy chain and a light chain or a heavy chain variable region and a light chain variable region. In a specific aspect, durvalumab or an antigen-binding fragment thereof for use in the methods provided herein comprises a light chain variable region comprising the amino acid sequence of SEQ ID NO: 1 and a heavy chain variable region comprising the amino acid sequence of SEQ ID NO: 2. In a specific aspect, durvalumab or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOs: 3-5, and wherein the light chain variable region comprises the Kabat-defined CDR1, CDR2, and CDR3 sequences of SEQ ID NOs: 6-8. Those of ordinary skill in the art would easily be able to identify Chothia-defined. Abm-defined or other CDR definitions known to those of ordinary skill in the art. In a specific aspect, durvalumab or an antigen-binding fragment thereof for use in the methods provided herein comprises the variable heavy chain and variable light chain CDR sequences of the 2.14H9OPT antibody as disclosed in U.S. Pat. Nos. 8,779,108 and 9,493,565, which are herein incorporated by reference in their entirety.

Patients with late stage (III or IV) locally advanced, unresectable NSCLC, who have not progressed following definitive chemoradiation therapy are administered an anti-PD-1 or anti-PD-L1 antibody, such as durvalumab, or an antigen-binding fragment thereof. Durvalumab or an antigen-binding fragment thereof can be administered once every two weeks while providing benefit to the patient. In further aspects the patient is administered additional follow-on doses. Follow-on doses can be administered at various time intervals depending on the patient's age, weight, clinical assessment, tumor burden, and/or other factors, including the judgment of the attending physician.

In aspects, multiple doses of durvalumab or an antigen-binding fragment thereof are administered to the patient. In some aspects, at least three doses, at least four doses, at least five doses, at least six doses, at least seven doses, at least eight doses, at least nine doses, at least ten doses, at least fifteen doses, or at least twenty six doses (i.e., a full year of treatment) or more can be administered to the patient. In some aspects, durvalumab or an antigen-binding fragment thereof is administered every two weeks, over a two week period, over a four-week treatment period, over a six-week treatment period, over an eight-week treatment period, over a twelve-week treatment period, over a twenty-four-week treatment period, or over a one-year or more treatment period.

In certain aspects, the interval between doses can be every three weeks. In certain aspects, the interval between doses can be every four weeks. In further aspects, the intervals between doses can be every two months (e.g., during a maintenance phase).

In certain aspects, the dosing intervals can also be about every 14 days or about every 21 days. In some aspects, “about” every 14 days or “about” every 21 days indicates 14 days+/−2 days or 21 days+/−2 days. In some aspects, administration of durvalumab is about every 14 to 21 days.

In certain aspects the patient is administered one or more doses of an anti-PD-L1 antibody, wherein the dose is a fixed dose of 1200 mg.

The amount of durvalumab or an antigen-binding fragment thereof to be administered to the patient may be adjusted and can depend on various parameters, such as the patient's age, weight, clinical assessment, tumor burden and/or other factors, including the judgment of the attending physician. In some aspects, the dose is a fixed dose.

In certain aspects the patient is administered one or more doses of durvalumab wherein the dose is about 1 mg/kg. In certain aspects the patient is administered one or more doses of durvalumab wherein the dose is about 3 mg/kg. In certain aspects the patient is administered one or more doses of durvalumab wherein the dose is about 10 mg/kg. In certain aspects the patient is administered one or more doses of durvalumab wherein the dose is about 15 mg/kg. In certain aspects the patient is administered one or more doses of durvalumab wherein the dose is about 20 mg/kg.

In certain aspects the patient is administered one or more doses of durvalumab wherein the dose is a fixed dose of 1500 mg.

In certain aspects the patient is administered at least two doses of durvalumab or an antigen-binding fragment thereof, once every two weeks, wherein the dose is about 10 mg/kg. In further aspects, the patient is administered a 10 mg/kg dose of durvalumab every two weeks for up to year or more.

In certain aspects, the patient is administered 1500 mg of durvalumab every four weeks.

In certain aspects, administration of durvalumab or an antigen-binding fragment thereof according to the methods provided herein is through parenteral administration. For example, durvalumab or an antigen-binding fragment thereof can be administered by intravenous infusion or by subcutaneous injection. In some aspects, the administration is by intravenous infusion.

In certain aspects, durvalumab or an antigen-binding fragment thereof is administered according to the methods provided herein in combination or in conjunction with additional cancer therapies. Such therapies include, without limitation, chemotherapeutic agents such as Vemurafenib, Erlotinib, Afatinib, Cetuximab, Carboplatin, Bevacizumab, Erlotinib, or Pemetrexed, or other chemotherapeutic agents, as well radiation or any other anti-cancer treatments.

The methods provided herein may provide for additional clinical benefits beyond those specifically identified and illustrated by the data including, for example, decrease tumor size, retard tumor growth or maintain a steady state. In certain aspects the reduction in tumor size can be significant based on appropriate statistical analyses. A reduction in tumor size can be measured by comparison to the size of patient's tumor at baseline, against an expected tumor size, against an expected tumor size based on a large patient population, or against the tumor size of a control population. In certain aspects provided herein, the administration of durvalumab can reduce a tumor size by at least 25%, at least 50%, or at least 75%.

The methods provided herein can decrease or retard tumor growth. In some aspects the reduction or retardation can be statistically significant. A reduction in tumor growth can be measured by comparison to the growth of patient's tumor at baseline, against an expected tumor growth, against an expected tumor growth based on a large patient population, or against the tumor growth of a control population.

According to the methods provided herein, administration of v or an antigen-binding fragment thereof can result in desirable pharmacokinetic parameters. Total drug exposure can be estimated using the “area under the curve” (AUC). “AUC (tau)” refers to AUC until the end of the dosing period, whereas “AUC (inf)” refers to the AUC until infinite time. The administration can produce AUC (tau) of about 100 to about 2,500 d·μg/mL. The administration can produce a maximum observed concentration (Cmax) of about 15 to about 350 μg/mL. The half-life of the durvalumab or the antigen-binding fragment thereof can be about 5 to about 25 days. In addition, the clearance of the durvalumab or the antigen-binding fragment thereof can be about 1-10 ml/day/kg.

As provided herein, durvalumab or an antigen-binding fragment thereof can also decrease free PD-L1 levels. Free PD-L1 refers to PD-L1 that is not bound (e.g., by durvalumab). In some aspects, PD-L1 levels are reduced by at least 80%. In some aspects, PD-L1 levels are reduced by at least 90%. In some aspects, PD-L1 levels are reduced by at least 95%. In some aspects, PD-L1 levels are reduced by at least 99%. In some aspects, PD-L1 levels are eliminated following administration of durvalumab or an antigen-binding fragment thereof. In some aspects, administration of durvalumab or an antigen-binding fragment thereof reduces the rate of increase of PD-L1 levels as compared, e.g., to the rate of increase of PD-L1 levels prior to the administration of durvalumab or an antigen-binding fragment thereof.

The practice of the methods disclosed herein employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991).

The following examples provide an illustration of some of the aspects and aspects described above, and are not intended to limit the scope of the claimed invention.

### EXAMPLES

### Example 1: Clinical Assessment of Durvalumab in the Treatment of Locally Advanced, Stage III, Unresectable NSCLC

This example provides results from an interim analysis of the randomized, double-blind, international, phase 3 PACIFIC study (ClinicalTrials.gov number NCT02125461) comparing durvalumab versus placebo as consolidation therapy in patients with stage III, locally advanced, unresectable NSCLC, whose disease had not progressed following platinum-based cCRT. This is the first randomized phase 3 study to evaluate immune checkpoint blockade in this setting.

### Patients

Eligible patients had histologically- or cytologically-documented stage III, locally advanced, unresectable NSCLC per the International Association for the Study of Lung Cancer Staging Manual in Thoracic Oncology (v7), had received ≥2 cycles (defined per local practice) of platinum-based chemotherapy (containing etoposide, vinblastine, vinorelbine, a taxane [paclitaxel or docetaxel], or pemetrexed) concurrently with definitive radiation therapy (54 Gy to 66 Gy), in which the mean lung dose must have been <20 Gy and/or V20 must have been <35%, and had not progressed following this treatment. Patients were aged ≥18 years, had a World Health Organization (WHO) PS 0 or 1, an estimated life expectancy 2 weeks, and had completed the last dose of radiation within 1 to 14 days before randomization (changed to 1 to 42 days, following a protocol amendment).

Key exclusion criteria include prior exposure to anti-PD-1 or anti-PD-L1 antibodies; receipt of immunotherapy or investigational drug within 4 weeks before the first dose (6 weeks in the case of monoclonal antibodies); active or prior autoimmune disease (within the past 2 years) or history of primary immunodeficiency; evidence of uncontrolled, concurrent illness; evidence of ongoing or active infections; unresolved toxicity from previous CRT>grade 2 (based on Common Terminology Criteria for Adverse Event [CTCAE]); ≥grade 2 pneumonitis from prior CRT.

### Design and Treatments

Patients were randomized within 1-42 days post cCRT in a 2:1 ratio to receive durvalumab 10 mg/kg intravenously or matching placebo every two weeks (q2w) as consolidation therapy for up to 12 months. Patients were stratified by age (<65 or ≥65 years), sex and smoking history (current/former smoker versus non-smoker). Study drug administration commenced following randomization on day 1, once the patient was confirmed eligible. The study drug was discontinued if there was confirmed disease progression, initiation of alternative anticancer therapy, unacceptable toxicity or consent withdrawal. Patients could be treated through progression (unless they had rapid tumor progression or symptomatic progression requiring urgent medical intervention) and re-treated if they had achieved disease control at end of the 12 months but progressed during follow-up.

### Endpoints and Assessments

The co-primary endpoints were PFS (according to Response Evaluation Criteria In Solid Tumors [RECIST] v1.1, as assessed by Blinded Independent Central Review [BICR]), and overall survival (OS). PFS was defined as the time from randomization to the date of the first documented event of tumor progression or death in the absence of progression, and OS was defined as the time from randomization until death (any cause). PFS was assessed by investigators, according to RECIST v1.1 as a pre-defined sensitivity analysis.

Secondary endpoints were the proportion of patients alive and progression-free at 12 and 18 months, objective response rate (ORR), duration of response (DoR), and time to death or distant metastasis (TTDM), all per BICR, and OS at 24 months, safety and tolerability (graded using CTCAE v4.03), health-related quality of life, pharmacokinetics and immunogenicity. Efficacy was assessed every 8 weeks for the first 12 months and every 12 weeks thereafter. All reported efficacy endpoints are for treatment with durvalumab or placebo only, i.e. they were not aggregate endpoints that included prior cCRT therapy.

Patients provided an optional archived tumor tissue sample for PD-L1 testing; however, enrollment was not restricted to any PD-L1 expression level thresholds.

### Statistical Analysis

The study was to be considered positive (a success) if analyses of either of the two co-primary endpoints, PFS or OS, were statistically significant. Approximately 702 patients were needed for 2:1 randomization to obtain 458 PFS events for the primary PFS analysis and 491 OS events for the primary OS analysis. It was estimated that the study would have at least 95% power to detect a PFS FIR of 0.67 and at least 85% power to detect an OS HR of 0.73, based on a log-rank test with a two-sided significance level of 2.5% for each co-primary endpoint. An interim analysis of PFS was planned when approximately 367 events had occurred. At this interim analysis, the PFS effects were estimated using the Kaplan-Meier method. The between-arm comparisons of PFS were performed using the log-rank test, stratified by age, sex and smoking history. Sensitivity analyses for PFS included the assessment of evaluation bias, evaluation time bias and attrition bias in the determination of progression, and adjustment for different covariates in the estimation of PFS effect. Response rates were estimated using the Clopper-Pearson method and compared using the fisher's exact test. Efficacy was assessed in the intent-to-treat population; safety was assessed in the as-treated population.

An external independent data monitoring committee (IDMC) is assessing ongoing safety and assessed the interim efficacy analyses.

### Patients and Treatment

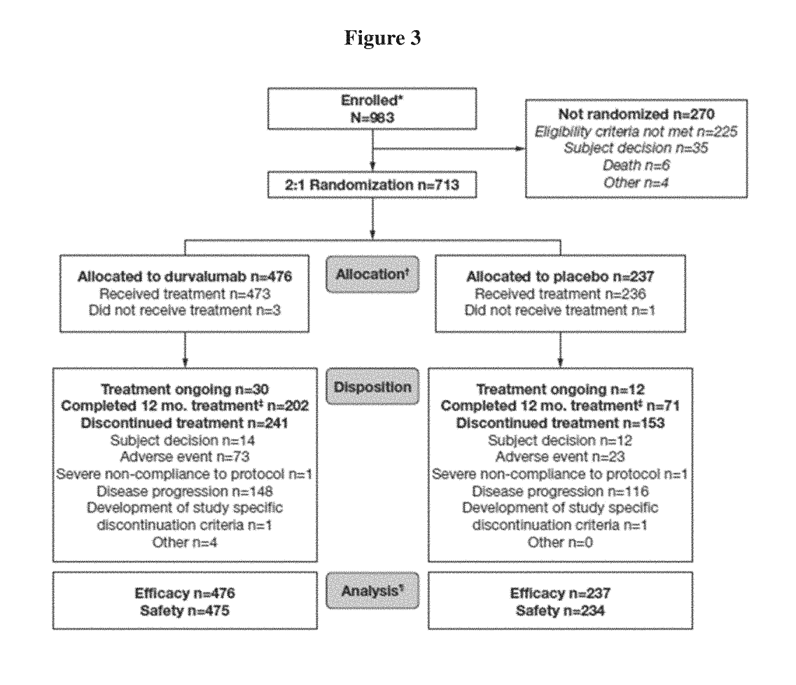

Between May 2014 and April 2016, 709 of 713 randomized patients (99%) received ≥1 dose of study drug as consolidation therapy (473 patients received durvalumab and 236 received placebo; FIG. 3). Baseline characteristics were well balanced between the two treatment groups (Table 1).

TABLE 1
Baseline Characteristics, Stratification Factors and Prior Therapy (Intention-to-Treat population).*

| | Durvalumab | | Placebo | | Total | |
|---|---|---|---|---|---|---|
| | (N = 476) | | (N = 237) | | (N = 713) | |
| Age - yr | | | | | | |
| Median (range) | 64 | (31-84) | 64 | (23-90) | 64 | (23-90) |
| Age category - no. (%) | | | | | | |
| ≥65 | 215 | (45.2) | 107 | (45.1) | 322 | (45.2) |
| Sex - no. (%) | | | | | | |
| Male | 334 | (70.2) | 166 | (70.0) | 500 | (70.1) |
| Female | 142 | (29.8) | 71 | (30.0) | 213 | (29.9) |
| Race - no. (%) | | | | | | |
| White | 337 | (70.8) | 157 | (66.2) | 494 | (69.3) |
| Black or African-American | 12 | (2.5) | 2 | (0.8) | 14 | (2.0) |
| Asian | 120 | (25.2) | 72 | (30.4) | 192 | (26.9) |
| Other | 6 | (1.3) | 6 | (1.3) | 12 | (1.68) |
| Not reported | 1 | (0.2) | 0 | | 1 | (0.1) |
| Disease stage | | | | | | |
| IIIA | 252 | (52.9) | 125 | (52.7) | 377 | (52.9) |
| IIIB | 212 | (44.5) | 107 | (45.1) | 319 | (44.7) |
| Other | 12 | (2.5) | 5 | (2.1) | 17 | (2.4) |
| WHO performance- status score - no. (%)^{†} | | | | | | |
| 0 | 234 | (49.2) | 114 | (48.1) | 348 | (48.8) |
| 1 | 240 | (50.4) | 122 | (51.5) | 362 | (50.8) |
| Not reported | 2 | (0.4) | 1 | (0.4) | 3 | (0.4) |
| Histology - no. (%) | | | | | | |
| Squamous | 224 | (47.1) | 102 | (43.0) | 326 | (45.7) |
| Non-squamous | 252 | (52.9) | 135 | (57.0) | 387 | (54.3) |
| Smoking status - no. (%) | | | | | | |
| Current smoker | 79 | (16.6) | 38 | (16.0) | 117 | (16.4) |
| Former smoker | 354 | (74.4) | 178 | (75.1) | 532 | (74.6) |
| Never smoked | 43 | (9.0) | 21 | (8.9) | 64 | (9.0) |
| Geographic region - no. (%) | | | | | | |
| North America | 144 | (30.3) | 67 | (28.3) | 211 | (29.6) |
| South America | 6 | (1.3) | 0 | | 6 | (0.8) |
| Europe | 217 | (45.6) | 102 | (43.0) | 319 | (44.7) |
| Asia | 109 | (22.9) | 68 | (28.7) | 177 | (24.8) |
| Prior radiotherapy - no. (%) | | | | | | |
| <54 Gy | 3 | (0.6) | 0 | | 3 | (0.6) |
| ≥54-≤66 Gy | 442 | (92.9) | 217 | (91.6) | 659 | (92.4) |
| >66-≤74 Gy | 30 | (6.3) | 19 | (8.0) | 49 | (6.9) |
| >74 Gy | 0 | | 0 | | 0 | |
| Missing^{‡} | 1 | (0.2) | 1 | (0.4) | 2 | (0.3) |
| Number of previous chemotherapy regimens - no. (%) | | | | | | |
| 1 | 444 | (93.3) | 224 | (94.5) | 668 | (93.7) |
| 2 | 32 | (6.7) | 13 | (5.5) | 45 | (6.3) |
| Prior chemotherapy - no. (%)^{£} | | | | | | |
| Adjuvant | 3 | (0.6) | 1 | (0.4) | 4 | (0.6) |
| Induction chemotherapy | 123 | (25.8) | 68 | (28.7) | 191 | (26.8) |
| Definitive | 475 | (99.8) | 236 | (99.6) | 711 | (99.7) |
| Best response to previous cCRT - no. (%) | | | | | | |
| Complete response | 9 | (1.9) | 7 | (3.0) | 16 | (2.2) |
| Partial response | 232 | (48.7) | 111 | (46.8) | 343 | (48.1) |
| Stable disease | 222 | (46.6) | 114 | (48.1) | 336 | (47.1) |
| Progression | 2 | (0.4) | 0 | | 2 | (0.3) |
| Non-evaluable | 9 | (1.9) | 4 | (1.7) | 13 | (1.8) |
| Not applicable | 2 | (0.4) | 1 | (0.4) | 3 | (0.4) |

*The intention-to-treat population included all patients who underwent randomization. There were no statistically significant (P < 0.05) between-group differences in the baseline characteristics listed here. Percentages may not total 100 because of rounding.
^{†}World Health Organization (WHO) performance status (PS) scores range from 0 to 5, with 0 indicating no symptoms and higher scores indicating increased disability
^{‡}For the two patients with missing data, the biologically effective radiotherapy dose could not be calculated, primarily because their radiotherapy treatment planning data were neither collected nor accessible.
^{£}Patients may have received prior chemotherapy in more than one context.

The median age of all patients was 64 years, and the majority were men (70%) and current/former smokers (91%); 46% of patients had squamous histology. Previous chemotherapies were also well balanced; 55.9% and 54.4% of patients in the durvalumab and placebo groups, respectively, had previously received a cisplatin-based definitive regimen, and 41.8% and 43.0% had received a carboplatin-based regimen (Table 2). In addition, 25.8% and 28.7% had received induction chemotherapy prior to definitive cCRT.

TABLE 2
Prior Definitive Chemotherapy Regimens (Intention-to-Treat population).

| | Durvalumab | | Placebo | | Total | |
|---|---|---|---|---|---|---|
| | (N = 476) | | (N = 237) | | (N = 713) | |
| Total - no. (%) | 473 | (99.4) | 236 | (99.6) | 709 | (99.4) |
| Cisplatin* | 266 | (55.9) | 129 | (54.4) | 395 | (55.4) |
| Cisplatin + etoposide | 106 | (22.3) | 49 | (20.7) | 155 | (21.7) |
| Cisplatin + vinorelbine | 77 | (16.2) | 34 | (14.3) | 111 | (15.6) |
| Cisplatin + vinorelbine ditartrate | 26 | (5.5) | 14 | (5.9) | 40 | (5.6) |
| Cisplatin + docetaxel | 26 | (5.5) | 8 | (3.4) | 34 | (4.8) |
| Cisplatin + paclitaxel | 13 | (2.7) | 15 | (6.3) | 28 | (3.9) |
| Cisplatin + pemetrexed | 11 | (2.3) | 5 | (2.1) | 16 | (2.2) |
| Cisplatin + nab-paclitaxel | 1 | (0.2) | 0 | | 1 | (0.1) |
| Cisplatin + vinblastine | 1 | (0.2) | 0 | | 1 | (0.1) |
| Cisplatin + other | 1 | (0.2) | 0 | | 1 | (0.1) |
| Carboplatin^{†} | 199 | (41.8) | 102 | (43.0) | 301 | (42.2) |
| Carboplatin + paclitaxel | 158 | (33.2) | 84 | (35.4) | 242 | (33.9) |
| Carboplatin + vinorelbine | 8 | (1.7) | 4 | (1.7) | 12 | (1.7) |
| Carboplatin + etoposide | 8 | (1.7) | 2 | (0.8) | 10 | (1.4) |
| Carboplatin + vinorelbine ditartrate | 7 | (1.5) | 5 | (2.1) | 12 | (1.7) |
| Carboplatin + pemetrexed | 7 | (1.5) | 4 | (1.7) | 11 | (1.5) |
| Carboplatin + docetaxel | 2 | (0.4) | 1 | (0.4) | 3 | (0.4) |
| Carboplatin + nab-paclitaxel | 2 | (0.4) | 0 | | 2 | (0.3) |
| Carboplatin + pemetrexed disodium | 1 | (0.2) | 0 | | 1 | (0.1) |
| Carboplatin + other | 2 | (0.4) | 1 | (0.4) | 3 | (0.4) |
| Cisplatin/carboplatin | 8 | (1.7) | 5 | (2.1) | 13 | (1.8) |
| Cisplatin/carboplatin + vinorelbine | 2 | (0.4) | 1 | (0.4) | 3 | (0.4) |
| Cisplatin/carboplatin + etoposide | 2 | (0.4) | 0 | | 2 | (0.3) |
| Cisplatin/carboplatin + pemetrexed | 1 | (0.2) | 1 | (0.4) | 2 | (0.3) |
| Cisplatin/carboplatin + docetaxel | 1 | (0.2) | 0 | | 1 | (0.1) |
| Cisplatin/carboplatin + vinorelbine ditartrate | 1 | (0.2) | 0 | | 1 | (0.1) |
| Cisplatin/carboplatin + other | 1 | (0.2) | 3 | (1.3) | 4 | (0.6) |

*Cisplatin alone was received by 4 patients in each group (0.8% and 1.7% in the durvalumab and placebo groups, respectively).
^{†}Carboplatin alone was received by 4 patients (0.8%) in the durvalumab group and 1 patient (0.4%) in the placebo group.

Based on pre-cCRT archived tumor samples (which were not re-assessed after cCRT), ≥25% PD-L1 expression on tumor cells (TC≥25%) occurred in 22% of patients (24% in the durvalumab group and 19% in the placebo group) and TC<25% occurred in 41% (39% in the durvalumab group and 44% in the placebo group); 37% of patients had unknown PD-L1 status (Table 3). EGFR mutations were observed in 6.0% of patients (6.1% in the durvalumab group and 5.9% in the placebo group), whereas 67.3% of patient's tumors were EGFR negative or wild-type (66.2% in the durvalumab group and 69.6% in the placebo group); EGFR mutation status was unknown in 27.7% and 24.5%, respectively (Table 3). There were no statistically significant (P<0.05) between-group differences in either PD-L1 expression or EGFR mutation status.

TABLE 3
Prevalence by PD-L1 Expression and EGFR Mutation
Status (Intention-to-Treat Population).*

| | Durvalumab | Placebo | |
|---|---|---|---|
| | (N = 476) | (N = 237) | |
| PD-L1 status - no. (%) | | | |
| TC < 25% | 187 (39.3) | 105 | (44.3) |
| TC ≥ 25% | 115 (24.2) | 44 | (18.6) |
| Unknown^{†} | 174 (36.6) | 88 | (37.1) |
| EGFR mutation status - no. (%) | | | |
| Positive | 29 (6.1) | 14 | (5.9) |
| Negative | 315 (66.2) | 165 | (69.6) |
| Unknown^{†} | 132 (27.7) | 58 | (24.5) |

*There were no statistically significant (P < 0.05) between-group differences in either PD-L1 expression or EGFR mutation status.
^{†}No sample collected or no valid test result.
TC ≥ 25%, ≥25% PD-L1 expression on tumor cells; TC < 25%, <25% PD-L1 expression on tumor cells.

Within data cutoff time for this analysis, overall median follow up was 14.5 months (range 0.2-29.9). The median number of infusions received was 20 (range 1-27) in the durvalumab group and 14 (range 1-26) in the placebo group; 6.3% and 5.1% were still receiving treatment at the data cutoff (Table 4).

TABLE 4
Patient Disposition.

| | Durvalumab | | Placebo | |
|---|---|---|---|---|
| | (N = 476) | | (N = 237) | |
| Received treatment - no. (%)* | 473 | (99.4) | 236 | (99.6) |
| Treatment ongoing at data cutoff - | 30 | (6.3) | 12 | (5.1) |
| no. (%)^{†} | | | | |
| Completed 12 months treatment - | 202 | (42.7) | 71 | (30.1) |
| no. (%)^{†} | | | | |
| Retreated after 12 months - no. (%)^{†,‡} | 6 | (1.3) | 6 | (2.5) |
| Discontinued study treatment - no. (%)^{†} | 241 | (51.0) | 153 | (64.8) |
| Subject decision | 14 | (3.0) | 12 | (5.1) |
| Adverse event | 73 | (15.4) | 23 | (9.7) |
| Severe non-compliance to protocol | 1 | (0.2) | 1 | (0.4) |
| Condition under investigation worsened | 148 | (31.3) | 116 | (49.2) |
| Development of study specific | 1 | (0.2) | 1 | (0.4) |
| discontinuation criteria | | | | |
| Other | 4 | (0.8) | 0 | |
| Ongoing at data cutoff - no. (%)^{†} | 346 | (73.2) | 144 | (61.0) |
| Discontinued study - no. (%)^{†,} | 121 | (25.6) | 92 | (39.0) |
| Received subsequent therapy after | 145 | (30.5) | 102 | (43.0) |
| discontinuation - no. (%)* | | | | |

*Percentages are calculated based on the number of patients in the full analysis set.
^{†}Percentages are calculated based on the number of patients who received treatment.
^{‡}Patients who achieved disease control at the end of 12 months but progressed during follow-up and, as allowed by the protocol, were retreated with the same blinded study treatment.

### Efficacy

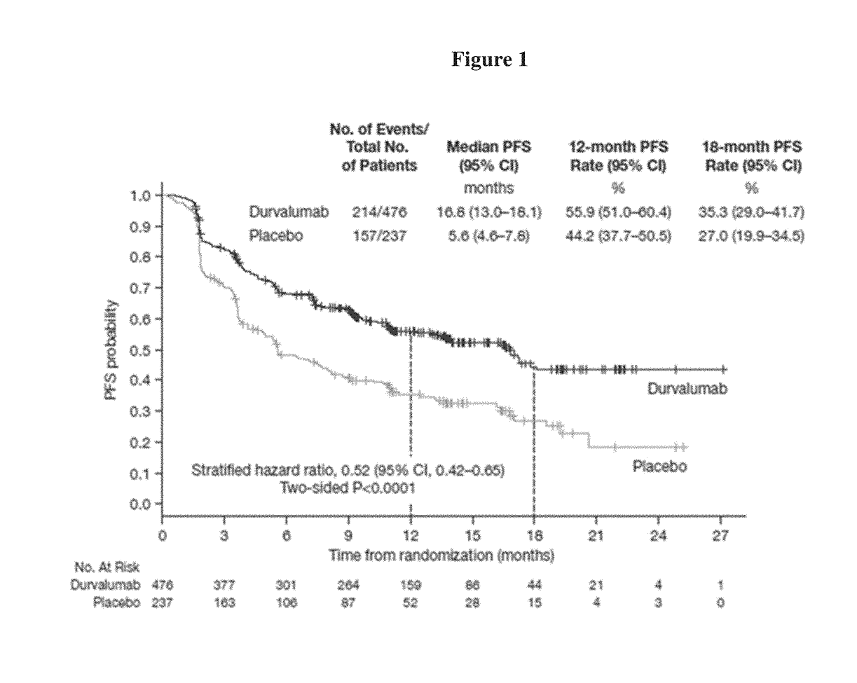

Median PFS from randomization, as assessed by BICR, was 16.8 months (95% confidence interval [CI], 13.0-18.1) with durvalumab versus 5.6 months (95% CI, 4.6-7.8) with placebo (stratified hazard ratio [HR] for disease progression or death, 0.52; 95% CI, 0.39-0.70; two-sided P<0.0001; FIG. 1). The 12-month PFS rate was 55.9% (95% CI, 51.0-60.4) with durvalumab and 35.3% (95% CI, 29.0-41.7) with placebo, and the 18-month PFS rate was 44.2% (95% CI, 37.7-50.5) and 27.0% (95% CI, 19.9-34.5), respectively. PFS results were robust and consistent across all pre-specified sensitivity analyses, including PFS by investigator assessment (stratified HR, 0.61; 95% CI, 0.50-0.76; two-sided P<0.0001).

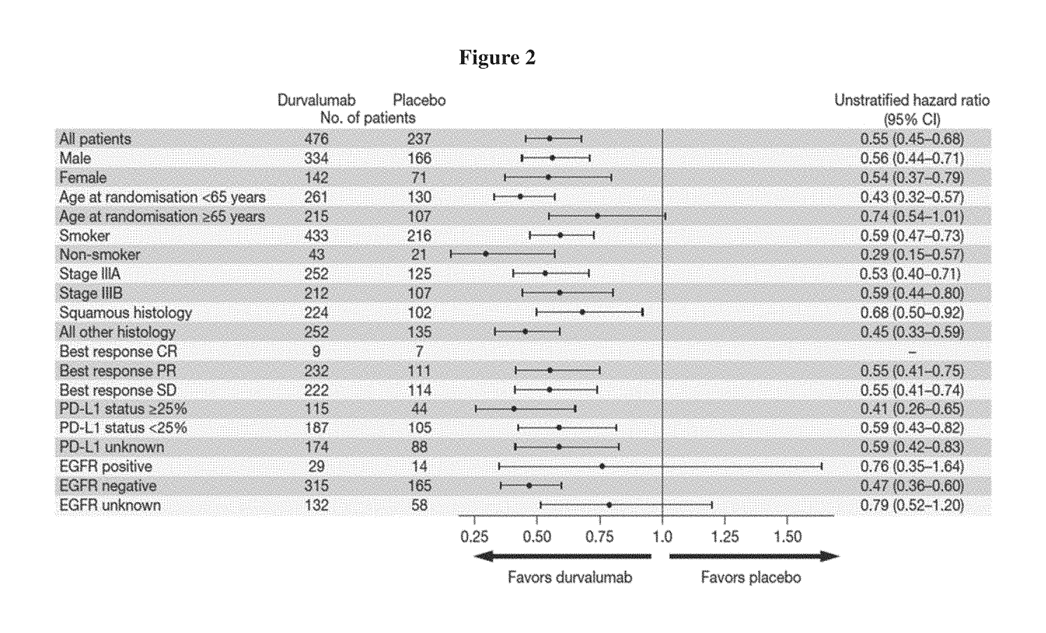

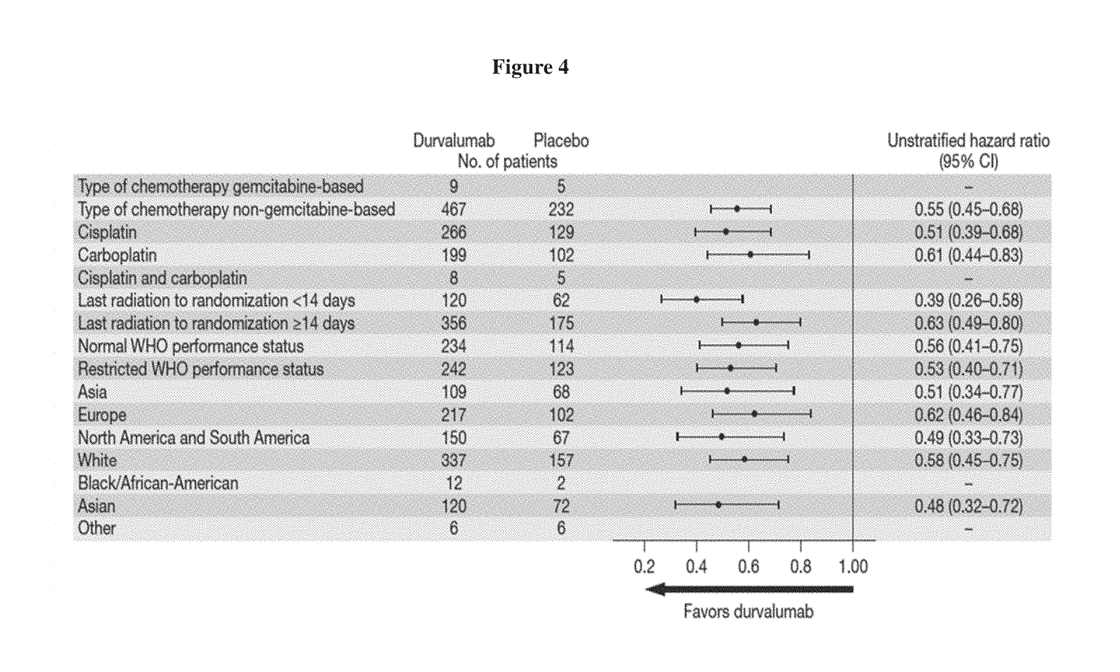

PFS benefit with durvalumab was consistently observed across all pre-specified subgroups, as defined by patient demographics, baseline clinicopathologic features, and response to prior treatment (FIG. 2; additional non-prognostic factors presented in FIG. 4). Notably, PFS benefit with durvalumab was observed irrespective of pre-cCRT PD-L1 expression (HR, 0.59; 95% CI, 0.43-0.82 for TC<25%, and HR, 0.41, 95% CI: 0.26-0.65 for TC≥25%). PFS benefit was also evident in non-smokers and patients with EGFR mutations.

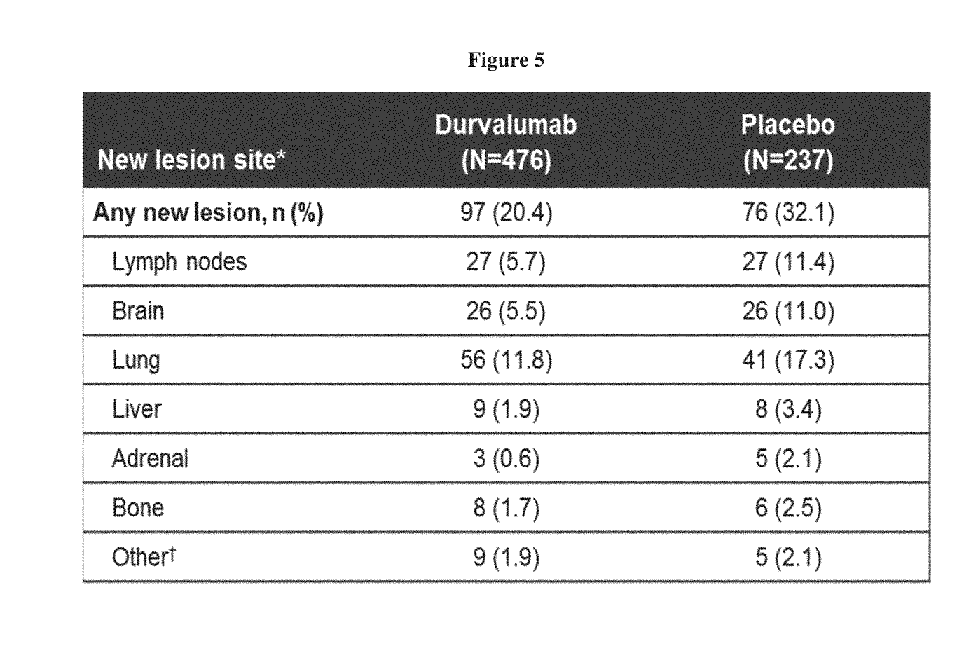

Median TTDM was 23.2 months (95% CI, 23.2-NR) with durvalumab versus 14.6 months (95% CI, 10.6-18.6) with placebo (HR, 0.52; 95% CI, 0.39-0.69; two-sided P<0.0001; FIG. 5). In addition, the frequency of new lesions, as assessed by BICR, was 20.4% with durvalumab and 32.1% with placebo, with lower incidences of new brain metastases with durvalumab (5.5% vs 11.0%, respectively) (Table 5).

TABLE 5
Incidence of New Lesions in the Intention-
to-Treat Population (BICR).*

| | |
|---|---|
| Durvalumab | Placebo |
| (N = 476) | (N = 237) |

| New lesion site^{†} | number of patients (percent) | | |
|---|---|---|---|
| Any new lesion | 97 | (20.4) | 76 (32.1) |
| Lung | 56 | (11.8) | 41 (17.3) |
| Lymph nodes | 27 | (5.7) | 27 (11.4) |
| Brain | 26 | (5.5) | 26 (11.0) |
| Liver | 9 | (1.9) | 8 (3.4) |
| Bone | 8 | (1.7) | 6 (2.5) |
| Adrenal | 3 | (0.6) | 5 (2.1) |
| Other | 9 | (1.9) | 5 (2.1) |

*According to RECIST v1.1.
^{†}A patient may have had more than one new lesion site.
BICR = Blinded Independent Central Review; RECIST, Response Evaluation Criteria In Solid Tumors.

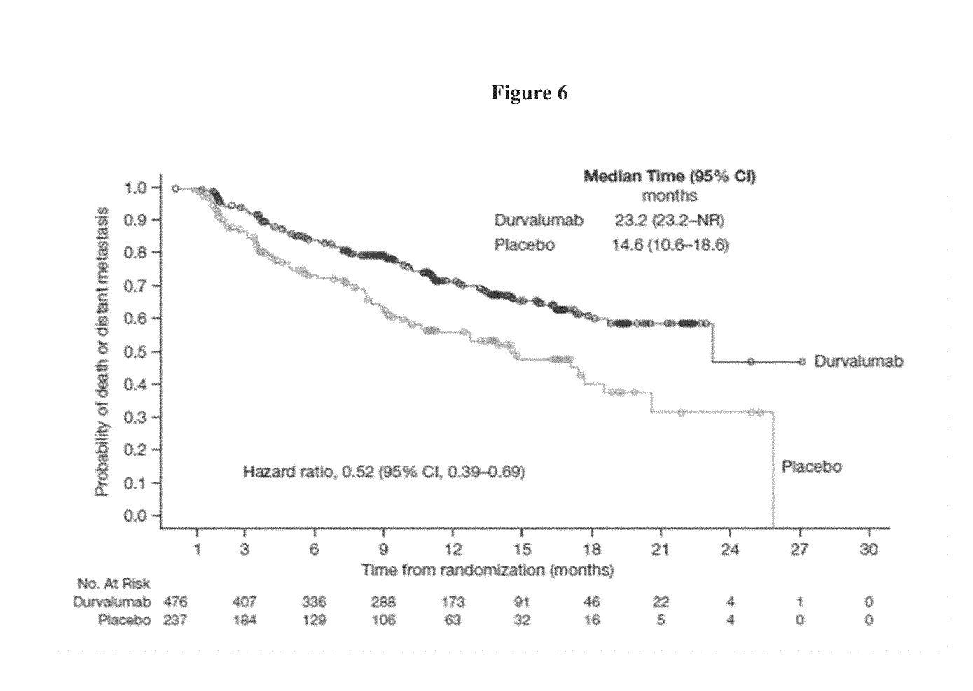

Treatment with durvalumab resulted in clinically meaningful improvement in ORR based on BICR (28.4% vs 16.0%, respectively; P<0.001) (Table 2); 16.5% and 27.7% of patients receiving durvalumab and placebo, respectively, experienced progressive disease (Table 6). Median DoR was not reached with durvalumab versus 13.8 months with placebo (Table 6; FIG. 6). Of the patients who responded to durvalumab, 72.8% had an ongoing response at both 12 and 18 months (Table 6).

TABLE 6
Antitumor Activity in the Intention-to-Treat Population (BICR).

| | Durvalumab | | Placebo | |
|---|---|---|---|---|
| | (N = 443)* | | (N = 213)* | |
| Confirmed objective | | | | |
| response | | | | |
| No. of patients | 126 | | 34 | |
| % of patients (95% CI) | 28.4 | (24.28, 32.89) | 16.0 | (11.31, 21.59) |
| P value | <0.001 | | | |
| Best overall response - | | | | |
| no. (%) | | | | |
| Complete response | 6 | (1.4) | 1 | (0.5) |
| Partial response | 120 | (27.1) | 33 | (15.5) |
| Stable disease | 233 | (52.6) | 119 | (55.9) |
| Progressive disease | 73 | (16.5) | 59 | (27.7) |
| Non-evaluable | 10 | (2.3) | 1 | (0.5) |
| Duration of response, | | | | |
| months | | | | |
| Median (95% CI) | NR | | 13.8 | (6.0, NR) |
| Ongoing response at data | | | | |
| cutoff - %^{†} | | | | |

| | | |
|---|---|---|
| At 12 months | 72.8 | 56.1 |
| At 18 months | 72.8 | 46.8 |

*Patients with measurable disease at baseline.
^{†}Percentages calculated by Kaplan-Meier method.
NR, not reached.

### Safety

Any-grade all-causality AEs occurred in 96.8% and 94.9% of patients receiving durvalumab and placebo, respectively (Table 7). Grade 3/4 AEs occurred in 29.9% and 26.1%, respectively. The most common grade 3/4 AE was pneumonia (4.4% vs 3.8%). Discontinuation due to AEs occurred in 15.4% and 9.8% of patients in the durvalumab and placebo groups, respectively. Death due to AEs occurred in 4.4% and 5.6%, respectively (Table 7). Treatment-related AEs are summarized in Table 8.

TABLE 7
All-Causality Adverse Events Reported in ≥10%
of Patients in Either Treatment Group.

| | Durvalumab | | | | Placebo | | | |
|---|---|---|---|---|---|---|---|---|
| | (N = 475) | | | | (N = 234) | | | |
| | Any | | Grade | | Any | | Grade | |
| | Grade* | | 3 or 4 | | Grade* | | 3 or 4 | |
| Event | number of patients with an event (percent) | | | | | | | |
| Any event | 460 | (96.8) | 142 | (29.9) | 222 | (94.9) | 61 | (26.1) |
| Cough | 168 | (35.4) | 2 | (0.4) | 59 | (25.2) | 1 | (0.4) |
| Pneumonitis/radiation | 161 | (33.9) | 16 | (3.4) | 58 | (24.8) | 6 | (2.6) |
| pneumonitis^{†} | | | | | | | | |
| Fatigue | 113 | (23.8) | 1 | (0.2) | 48 | (20.5) | 3 | (1.3) |
| Dyspnea | 106 | (22.3) | 7 | (1.5) | 56 | (23.9) | 6 | (2.6) |
| Diarrhea | 87 | (18.3) | 3 | (0.6) | 44 | (18.8) | 3 | (1.3) |
| Pyrexia | 70 | (14.7) | 1 | (0.2) | 21 | (9.0) | 0 | |
| Decreased appetite | 68 | (14.3) | 1 | (0.2) | 30 | (12.8) | 2 | (0.9) |
| Nausea | 66 | (13.9) | 0 | | 31 | (13.2) | 0 | |
| Pneumonia | 62 | (13.1) | 21 | (4.4) | 18 | (7.7) | 9 | (3.8) |
| Arthralgia | 59 | (12.4) | 0 | | 26 | (11.1) | 0 | |
| Pruritus | 58 | (12.2) | 0 | | 11 | (4.7) | 0 | |
| Rash | 58 | (12.2) | 1 | (0.2) | 17 | (7.3) | 0 | |
| Upper respiratory tract | 58 | (12.2) | 1 | (0.2) | 23 | (9.8) | 0 | |
| infection | | | | | | | | |
| Constipation | 56 | (11.8) | 1 | (0.2) | 20 | (8.5) | 0 | |
| Hypothyroidism | 55 | (11.6) | 1 | (0.2) | 4 | (1.7) | 0 | |
| Headache | 52 | (10.9) | 1 | (0.2) | 21 | (9.0) | 2 | (0.9) |
| Asthenia | 51 | (10.7) | 3 | (0.6) | 31 | (13.2) | 1 | (0.4) |
| Back pain | 50 | (10.5) | 1 | (0.2) | 27 | (11.5) | 1 | (0.4) |
| Musculoskeletal pain | 39 | (8.2) | 3 | (0.6) | 24 | (10.3) | 1 | (0.4) |
| Anemia | 36 | (7.6) | 14 | (2.9) | 25 | (10.7) | 8 | (3.4) |

*Grade 5 all-causality adverse events occurred in 21 patients (4.4%) receiving durvalumab (n = 4 [0.8%] with pneumonitis, n = 2 [0.4%] with cardiac arrest, and n = 1 each [0.2%] with the following: pneumonia, pneumonia bacterial, pneumonia pneumococcal, sepsis, septic shock, cardiomyopathy, cardiopulmonary failure, myocardial infarction, aortic dissection, dyspnea, emphysema, hemoptysis, respiratory distress, respiratory failure, radiation pneumonitis, right ventricular failure, brain natriuretic peptide increased, and unknown cause). Grade 5 all-causality adverse events occurred in 13 patients (5.6%) receiving placebo (n = 3 each [1.3%] with pneumonitis and pneumonia, and n = 1 each [0.4%] with the following: pneumonia streptococcal, West Nile viral infection, cardiac arrest, eosinophilic myocarditis, hemoptysis, intestinal obstructions, radiation pneumonitis and unknown cause).
^{†}Pneumonitis/radiation pneumonitis was assessed by investigators with subsequent review and adjudication by the study sponsor. In addition, pneumonitis, as reported in the table, is a grouped term, which includes acute interstitial pneumonitis, interstitial lung disease, pneumonitis, and pulmonary fibrosis.

TABLE 8
Treatment-Related Adverse Events Reported in ≥
75% of Patients in Either Treatment Group.

| | Durvalumab | | | | Placebo | | | |
|---|---|---|---|---|---|---|---|---|
| | (N = 475) | | | | (N = 234) | | | |
| | Any | | Grade | | Any | | Grade | |
| | Grade* | | 3 or 4 | | Grade* | | 3 or 4 | |
| Event | number of patients with an event (percent) | | | | | | | |
| Any event | 322 | (67.8) | 56 | (11.8) | 125 | (53.4) | 10 | (4.3) |
| Fatigue | 62 | (13.1) | 1 | (0.2) | 26 | (11.1) | 0 | |
| Hypo- | 50 | (10.5) | 1 | (0.2) | 1 | (0.4) | 0 | |
| thyroidism | | | | | | | | |
| Diarrhea | 46 | (9.7) | 2 | (0.4) | 19 | (8.1) | 2 | (0.9) |
| Pneumonitis | 43 | (9.1) | 6 | (1.3) | 8 | (3.4) | 2 | (0.9) |

| Rash | 37 | (7.8) | 1 | (0.2) | 13 | (5.6) | 0 |
|---|---|---|---|---|---|---|---|
| Pruritus | 33 | (6.9) | 0 | | 5 | (2.1) | 0 |
| Hyper- | 30 | (6.3) | 0 | | 3 | (1.3) | 0 |
| thyroidism | | | | | | | |
| Asthenia | 28 | (5.9) | 3 | (0.6) | 15 | (6.4) | 0 |
| Dyspnea | 28 | (5.9) | 3 | (0.6) | 8 | (3.4) | 0 |

| | | | | | | | |
|---|---|---|---|---|---|---|---|
| Decreased | 27 | (5.7) | 0 | 7 | (3.0) | 1 | (0.4) |
| appetite | | | | | | | |

| | | | | | | |
|---|---|---|---|---|---|---|
| Nausea | 26 | (5.5) | 0 | 14 | (6.0) | 0 |
| Cough | 25 | (5.3) | 0 | 4 | (1.7) | 0 |

*Grade 5 treatment-related adverse events occurred in 7 patients (1.5%) receiving durvalumab (n = 4 [0.8%] with pneumonitis and n = 1 each [0.2%] with the following: cardiomyopathy, right ventricular failure, respiratory distress, respiratory failure, brain natriuretic peptide increased, and radiation pneumonitis). Grade 5 treatment-related adverse events occurred in 3 patients (1.3%) receiving placebo (n = 2 [0.9%] with pneumonitis and n = 1 [0.4%] with unknown cause).

The most frequent AEs leading to discontinuation of durvalumab and placebo were pneumonitis/radiation pneumonitis (6.3% vs 4.3%) and pneumonia (1.1% vs 1.3%). Any-grade (grade 3/4) pneumonitis/radiation pneumonitis occurred in 33.9% versus 24.8% (3.4% vs 2.6%) and any-grade (grade 3/4) pneumonia occurred in 13.1% versus 7.7% (4.4% vs 3.8%) with durvalumab and placebo, respectively.

Any-grade AEs of special interest (AESIs), regardless of causality, were reported in 66.1% and 48.7% of patients in the durvalumab and placebo groups, respectively. The majority were grade 1/2, with grade≥3 incidences infrequent (<10%) in both treatment groups. The most frequent any-grade AESIs with durvalumab versus placebo were diarrhea (18.3% vs 18.8%), pneumonitis (12.6% vs. 7.7%), rash (12.2% vs 7.3%) and pruritus (12.2% vs. 4.7%). AESIs requiring concomitant treatment were reported in 42.1% and 17.1% of patients, respectively; treatments for AESIs included steroids (15.2% vs 6.8%), high dose steroids (8.8% vs 5.1%), endocrine therapy (11.6% vs 1.3%) and other immunosuppressants (0.4% of both groups).

Any-grade immune-mediated AEs, regardless of causality, were reported in 24.2% and 8.1% of patients receiving durvalumab and placebo, respectively; grade 3/4 immune-mediated AEs were reported in 3.4% and 2.6% of patients, respectively (Table 9). Treatments for immune-mediated AEs included systemic steroids (14.3% vs 5.6%), high dose steroids (8.2% vs 4.3%), endocrine therapy (10.7% vs 1.3%) and other immunosuppressants (0.4% of both groups).

TABLE 9
Any-Grade Immune-Mediated Adverse Events* Reported
in ≥1% of Patients in Either Treatment Group.

| | Durvalumab | | | | Placebo | | |
|---|---|---|---|---|---|---|---|
| | (N = 475) | | | | (N = 234) | | |
| | Any | | Grade | | Any | | Grade |
| | Grade^{†} | | 3 or 4 | | Grade^{†} | | 3 or 4 |
| Event | number of patients with an event (percent) | | | | | | |
| Any event | 115 | (24.2) | 16 | (3.4) | 19 | (8.1) | 6 (2.6) |
| Pneumonitis | 51 | (10.7) | 8 | (1.7) | 16 | (6.8) | 6 (2.6) |
| Hypothyroidism | 44 | (9.3) | 1 | (0.2) | 3 | (1.3) | 0 |
| Hyperthyroidism | 13 | (2.7) | 0 | | 0 | | 0 |
| Rash | 5 | (1.1) | 2 | (0.4) | 1 | (0.4) | 0 |

| | | | | | |
|---|---|---|---|---|---|
| Dermatitis | 5 | (1.1) | 0 | 0 | 0 |

*An adverse event of special interest requiring the use of systemic steroids or other immunosuppressants, and/or, for specific endocrine events, endocrine therapy, consistent with an immune-mediated mechanism of action, and where there is no clear alternate etiology.
^{†}Grade 5 immune-mediated AEs occurred in 4 patients (0.8%) receiving durvalumab and 3 patients (1.3%) receiving placebo.

Following an AE, patients may continue to receive treatment Durvalumab. Treatment methods are disclosed below in Table 10.

TABLE 10
Durvalumab Treatment and Dose Variations Following Adverse Events

| AdverseReaction | Severity | Dosage Modification |
|---|---|---|
| Pneumonitis | Grade 2 | Withhold dose until Grade 1 or resolved and |
| | | corticosteroid dose is less than or equal to |
| | | prednisone 10 mg per day (or equivalent). |
| | Grade 3 or 4 | Permanently discontinue |
| Hepatitis | For ALT or AST | Withhold dose until Grade 1 or resolved and |
| | greater than 3 but | corticosteroid dose is less than or equal to |
| | less than or equal to | prednisone 10 mg per day (or equivalent). |
| | 8 times the ULN or | |
| | Total bilirubin | |
| | greater than 1.5 but | |
| | less than or equal to | |
| | 5 times the ULN | |
| | ALT or AST greater | Permanently discontinue |
| | than 8 times the | |
| | ULN or total | |
| | bilirubin greater than | |
| | 5 times the ULN or | |
| | Concurrent ALT or | |
| | AST greater than 3 | |
| | times the ULN and | |
| | total bilirubin greater | |
| | than 2 times the | |
| | ULN with no other | |
| | cause | |
| Colitis or diarrhea | Grade 2 | Withhold dose until Grade 1 or resolved and |
| | | corticosteroid dose is less than or equal to |
| | | prednisone 10 mg per day (or equivalent). |
| | Grade 3 or 4 | Permanently discontinue |
| Hyperthyroidism | Grade 2-4 | Withhold dose until clinically stable |
| Adrenal insufficiency or | Grade 2-4 | Withhold dose until clinically stable |
| Hypophysitis/Hypopituitarism | | |
| Nephritis | For Creatinine | Withhold dose until Grade 1 or resolved and |
| | greater than 1.5 to 3 | corticosteroid dose is less than or equal to |
| | times the ULN | prednisone 10 mg per day (or equivalent). |
| | For Creatinine | Permanently discontinue |
| | greater than 3 times | |
| | the ULN | |
| Rash or dermatitis | Grade 2 for longer | Withhold dose until Grade 1 or resolved and |
| | than 1 week or | corticosteroid dose is less than or equal to |
| | Grade 3 | prednisone 10 mg per day (or equivalent). |
| | Grade 4 | Permanently discontinue |
| Infection | Grade 3 or 4 | Withhold dose until clinically stable |
| Infusion-related reactions | Grade 1 or 2 | Interrupt or slow the rate of infusion |
| | Grade 3 or 4 | Permanently discontinue |
| Other immune-mediated adverse | Grade 3 | Withhold dose until Grade 1 or resolved and |
| reactions | | corticosteroid dose is less than or equal to |
| | | prednisone 10 mg per day (or equivalent). |
| | Grade 4 | Permanently discontinue |
| Myocarditis | Grade 2 | Withhold dose until resolved and completion of |
| | | corticosteroid taper. |
| | Grade 3 or 4, or any | Permanently discontinue |
| | Grade with positive | |
| | biopsy | |
| Myositis/Polymyositis | Grade 2 or 3 | Withhold dose until Grade 1 or resolved and |
| | | corticosteroid dose is less than or equal to |
| | | prednisone 10 mg per day (or equivalent). |
| | | Permanently discontinue if adverse reaction does |
| | | not resolve to less than or equal to Grade 1 within |
| | | 30 days or if there are signs of respiratory |
| | | insufficiency. |
| | Grade 4 | Permanently discontinue |
| Persistent Grade 2 or 3 adverse | Grade 2 or 3 adverse | Permanently discontinue |
| reaction (excluding | reaction that does | |
| endocrinopathies) | not recover to Grade | |
| | 0 or 1 within 12 | |
| | weeks after last | |
| | IMFINZI dose | |
| Inability to taper corticosteroid | Inability to reduce to | Permanently discontinue |
| | less than or equal to | |
| | prednisone 10 mg | |
| | per day (or | |
| | equivalent) within | |
| | 12 weeks after the | |
| | last IMFINZI dose | |
| Recurrent Grade 3 or 4 adverse | Recurrent Grade 3 or | Permanently discontinue |
| reaction | 4 (severe or life- | |
| | threatening) adverse | |
| | reaction | |

As shown above, the methods were able to meet the co-primary endpoint of PFS. Durvalumab demonstrated a statistically significant and robust improvement in PFS of more than 11 months compared with placebo (HR 0.52; P<0.0001) in patients with locally advanced, unresectable NSCLC. The disclosure herein represents, the largest absolute PFS benefit shown to date with an immunotherapy in any cancer or setting, and it is especially notable that this was accomplished in a biomarker-unselected population. Patients with a PD-L1 expression status of TC<25% comprised a larger proportion of participants in this study than patients with TC≥25%. In addition, PFS improvement with durvalumab was demonstrated across all pre-specified subgroups, including patients who were unexpected to respond based on trials in the advanced or metastatic setting.

Outcomes with durvalumab were shown to be clinically meaningful, as evidenced by improvement in all secondary endpoints, such as the clinically meaningful improvement in ORR of 12% compared with placebo (P<0.001). In addition, responses with durvalumab were durable compared with placebo (median DoR was not reached vs 13.8 months, respectively). Durvalumab also had a favorable impact on the frequency of new metastases, including a lower incidence of new brain metastases.

Durvalumab had a favorable safety profile in this population, which was consistent with other immunotherapies and its known safety profile as monotherapy in patients with more severe disease (stage IIIB/IV NSCLC). Although the incidences of some all-causality AEs, including pneumonitis/radiation pneumonitis, were higher with both durvalumab and placebo in this study, this was not unexpected in this post-definitive-dose cCRT setting. In addition, pneumonitis/radiation pneumonitis with durvalumab was mostly low grade with the incidences of clinically important grade 3/4 events well balanced between the two treatment groups (3.4% vs 2.6%) and lower than that reported in other studies in the same setting. The favorable safety profile of durvalumab following cCRT shown here may have implications in other disease settings.

Pre-clinical evidence suggests that PD-1 expression may be upregulated in tumor cells following chemotherapy and/or radiotherapy thus dampening immune activation. Therefore, tumors may be more sensitive to anti-PD-L1 therapy after cCRT. The disclosure herein suggests that efficacy with durvalumab was observed irrespective of pre-cCRT PD-L1 expression status or type of platinum-doublet. Furthermore, Dovedi et al. have suggested that concomitant but not sequential administration of anti-PD-L1 treatment with fractionated radiotherapy may improve survival. However, the data disclosed herein clearly demonstrate a clinical benefit for sequential administration of durvalumab within 42 days following cCRT.

The data demonstrates a statistically significant and clinically meaningful improvement in PFS and a manageable safety profile with durvalumab following cCRT in stage III, unresectable NSCLC. These positive findings in an unselected patient population, irrespective of baseline pre-cCRT tumoral PD-L1 expression, suggest a potential new role for durvalumab in this setting and, more generally, suggest that additional clinical benefit could be achieved by using immunotherapies in combined modality therapy.

### Other Aspects

From the foregoing description, it will be apparent that variations and modifications may be made to the invention described herein to adopt it to various usages and conditions. Such aspects are also within the scope of the following claims.

The recitation of a listing of elements in any definition of a variable herein includes definitions of that variable as any single element or combination (or subcombination) of listed elements. The recitation of an aspect herein includes that aspect as any single aspect or in combination with any other aspects or portions thereof.

All patents and publications mentioned in this specification are herein incorporated by reference to the same extent as if each independent patent and publication was specifically and individually indicated to be incorporated by reference.

SEQUENCE LISTING

SEQ ID NO: 1
EIVLTQSPGTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIK
SEQ ID NO: 2
EVQLVESGGGLVQPGGSLRLSCAASGFTFSRYWMSWVRQAPGKGLEWVAN
IKQDGSEKYYVDSVKGRFTISRDNAKNSLYLQMNSLRAEDTAVYYCAREG
GWFGELAFDYWGQGTLVTVSS
SEQ ID NO: 3-VH CDR1
GFTFSRYWMS
SEQ ID NO: 4-VH CDR2
NIKQDGSEKYYVDSVKG
SEQ ID NO: 5-VH CDR3
EGGWFGELAFDY
SEQ ID NO: 6-VL CDR1
RASQRVSSSYLA
SEQ ID NO: 7-VL CDR2
DASSRAT
SEQ ID NO: 8-VL CDR3
QQYGSLPWT

What is claimed is:

1. A method of extending progression-free survival (PFS) in a patient with, unresectable non-small-cell lung cancer (NSCLC), the method comprising treating the patient with a human anti-PD-L1 antibody, wherein the patient is a stage III patient who has not progressed following definitive chemoradiation therapy.

2. The method of claim 1, wherein the chemoradiation therapy is platinum-based.

3. The method of claim 1 wherein the human anti-PD-L1 antibody comprises a light chain variable domain comprising the amino acid sequence of SEQ ID NO: 1 and a heavy chain variable domain comprising the amino acid sequence of SEQ ID NO: 2.

4. The method of claim 1, wherein the human anti-PD-L1 antibody comprises:
a VH CDR1 having the amino acid sequence of SEQ ID NO: 3; and
a VH CDR2 having the amino acid sequence of SEQ ID NO: 4; and
a VH CDR3 having the amino acid sequence of SEQ ID NO: 5; and
a VL CDR1 having the amino acid sequence of SEQ ID NO: 6; and
a VL CDR2 having the amino acid sequence of SEQ ID NO: 7; and
a VL CDR3 having the amino acid sequence of SEQ ID NO: 8.

5. The method of claim 1, wherein the human anti-PD-L1 antibody is durvalumab, avelumab, or atezolizumab.

6. The method of claim 1, wherein treatment with the human anti-PD-L1 antibody is 10 mg/kg, intravenously Q2W.

7. The method of claim 1, wherein PFS is increased by at least five months versus placebo.

8. The method of claim 1, wherein PFS is at least 13 months.

9. The method of claim 1, wherein the patient is PD-L1 (+).

10. The method of claim 1, wherein the patient is PD-L1 (−).

11. The method of claim 1, wherein the patient is EGFR mutation (+).

12. The method of claim 1, wherein the patient is EGFR mutation (−) or wild type.

13. A method of increasing the overall response rate (ORR) in a patient with, unresectable NSCLC, the method comprising treating the patient with a human anti-PD-L1 antibody, wherein the patient is at a stage III patient who has not progressed following definitive chemoradiation therapy.

14. The method of claim 13, wherein the chemoradiation therapy is platinum-based.

15. The method of claim 13 wherein the human anti-PD-L1 antibody comprises a light chain variable domain comprising the amino acid sequence of SEQ ID NO: 1 and a heavy chain variable domain comprising the amino acid sequence of SEQ ID NO: 2.

16. The method of claim 13, wherein the human anti-PD-L1 antibody comprises:
a VH CDR1 having the amino acid sequence of SEQ ID NO: 3; and
a VH CDR2 having the amino acid sequence of SEQ ID NO: 4; and
a VH CDR3 having the amino acid sequence of SEQ ID NO: 5; and
a VL CDR1 having the amino acid sequence of SEQ ID NO: 6; and
a VL CDR2 having the amino acid sequence of SEQ ID NO: 7; and
a VL CDR3 having the amino acid sequence of SEQ ID NO: 8.

17. The method of claim 13, wherein the human anti-PD-L1 antibody is durvalumab, avelumab, or atezolizumab.

18. The method of claim 13, wherein treatment with the human anti-PD-L1 antibody is 10 mg/kg, intravenously Q2W.

19. The method of claim 13, wherein the ORR is increased by east 12% versus placebo.

20. The method of claim 13, wherein the patient is PD-L1 (+).

21. The method of claim 13, wherein the patient is PD-L1 (−).

22. The method of claim 13, wherein the patient is EGFR mutation (+).

23. The method of claim 13, wherein the patient is EGFR mutation (−) or wild type.

24. A method of increasing the time to death or metastasis (TTDM) in a patient with, unresectable NSCLC, the method comprising treating the patient with a human anti-PD-L1 antibody, wherein the patient is at a stage III patient who has not progressed following definitive chemoradiation therapy.

25. The method of claim 24, wherein the chemoradiation therapy is platinum-based.

26. The method of claim 24 wherein the human anti-PD-L1 antibody comprises a light chain variable domain comprising the amino acid sequence of SEQ ID NO: 1 and a heavy chain variable domain comprising the amino acid sequence of SEQ ID NO: 2.

27. The method of claim 24, wherein the human anti-PD-L1 antibody comprises:
a VH CDR1 having the amino acid sequence of SEQ ID NO: 3; and
a VH CDR2 having the amino acid sequence of SEQ ID NO: 4; and
a VH CDR3 having the amino acid sequence of SEQ ID NO: 5; and
a VL CDR1 having the amino acid sequence of SEQ ID NO: 6; and
a VL CDR2 having the amino acid sequence of SEQ ID NO: 7; and
a VL CDR3 having the amino acid sequence of SEQ ID NO: 8.

28. The method of claim 24, wherein the human anti-PD-L1 antibody is durvalumab, avelumab, or atezolizumab.

29. The method of claim 24, wherein treatment with the human anti-PD-L1 antibody is 10 mg/kg, intravenously Q2W.

30. The method of claim 24, wherein the TDDM is increased by at least four months versus placebo.

31. A method of lowering incidences of brain metastasis in a patient with, unresectable NSCLC, the method comprising treating the patient with a human anti-PD-L1 antibody, wherein the patient is at a stage III patient who has not progressed following definitive chemoradiation therapy.

32. The method of claim 31, wherein the chemoradiation therapy is platinum-based.

33. The method of claim 31 wherein the human anti-PD-L1 antibody comprises a light chain variable domain comprising the amino acid sequence of SEQ ID NO: 1 and a heavy chain variable domain comprising the amino acid sequence of SEQ ID NO: 2.

34. The method of claim 31, wherein the human anti-PD-L1 antibody comprises:
a VH CDR1 having the amino acid sequence of SEQ ID NO: 3; and
a VH CDR2 having the amino acid sequence of SEQ ID NO: 4; and
a VH CDR3 having the amino acid sequence of SEQ ID NO: 5; and
a VL CDR1 having the amino acid sequence of SEQ ID NO: 6; and
a VL CDR2 having the amino acid sequence of SEQ ID NO: 7; and
a VL CDR3 having the amino acid sequence of SEQ ID NO: 8.

35. The method of claim 31, wherein the human anti-PD-L1 antibody is durvalumab, avelumab, or atezolizumab.

36. The method of claim 31, wherein treatment with the human anti-PD-L1 antibody is 10 mg/kg, intravenously Q2W.

37. The method of claim 31, wherein the incidences of brain metastasis is decreased by at least five months versus placebo.

38. A method treating a patient with stage III unresectable NSCLC, the method comprising treating the patient with a human anti-PD-L1 antibody, wherein the patient has not progressed following definitive chemoradiation therapy.

39. The method of claim 38, wherein the human anti-PD-L1 antibody comprises a light chain variable domain comprising the amino acid sequence of SEQ ID NO: 1 and a heavy chain variable domain comprising the amino acid sequence of SEQ ID NO: 2.

40. The method of claim 38, wherein the human anti-PD-L1 antibody comprises:
a VH CDR1 having the amino acid sequence of SEQ ID NO: 3; and
a VH CDR2 having the amino acid sequence of SEQ ID NO: 4; and
a VH CDR3 having the amino acid sequence of SEQ ID NO: 5; and
a VL CDR1 having the amino acid sequence of SEQ ID NO: 6; and
a VL CDR2 having the amino acid sequence of SEQ ID NO: 7; and
a VL CDR3 having the amino acid sequence of SEQ ID NO: 8.

41. The method of claim 38, wherein the anti-PD-L1 antibody is durvalumab, avelumab, or atezolizumab.

42. The method of claim 38, wherein treatment with the human anti-PD-L1 antibody is 10 mg/kg, intravenously Q2W.


SEQUENCE LISTING (10 sequences)

SEQ ID NO: 1 - protein, 108 aa, Artificial Sequence
  Description of Artificial Sequence Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 121 aa, Artificial Sequence
  Description of Artificial Sequence Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 3 - protein, 10 aa, Artificial Sequence
  Description of Artificial Sequence Synthetic peptide
GFTFSRYWMS

SEQ ID NO: 4 - protein, 17 aa, Artificial Sequence
  Description of Artificial Sequence Synthetic peptide
NIKQDGSEKYYVDSVKG

SEQ ID NO: 5 - protein, 12 aa, Artificial Sequence
  Description of Artificial Sequence Synthetic peptide
EGGWFGELAFDY

SEQ ID NO: 6 - protein, 12 aa, Artificial Sequence
  Description of Artificial Sequence Synthetic peptide
RASQRVSSSYLA

SEQ ID NO: 7 - protein, 7 aa, Artificial Sequence
  Description of Artificial Sequence Synthetic peptide
DASSRAT

SEQ ID NO: 8 - protein, 9 aa, Artificial Sequence
  Description of Artificial Sequence Synthetic peptide
QQYGSLPWT

SEQ ID NO: 9 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 10 - DNA, 3349 bp, Homo sapiens
ggcgcaacgctgagcagctggcgcgtcccgcgcggccccagttctgcgcagcttcccgaggctccgcaccagccgcgcttctgtccgcctgcagggcattccagaaagatgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagttaaaaaa